(19) United States
(12) Patent Application Publication
(10) Pub. No.: US 2026/0090715 A1
(43) Pub. Date: Apr. 2, 2026

(54) IMAGING SYSTEM FOR SIMULTANEOUS IMAGING OF OBJECTS

(71) Applicant: Washington University, St. Louis, MO (US)

(72) Inventors: Chao Zhou, St. Louis, MO (US); Aaron Adkins, St. Louis, MO (US); Senyue Hao, St. Louis, MO (US)

(21) Appl. No.: 19/346,272

(22) Filed: Sep. 30, 2025

Related U.S. Application Data
(60) Provisional application No. 63/701,849, filed on Oct. 1, 2024.

(51) Int. Cl. A61B 3/10; G06T 3/4038; G06T 7/00; G06T 7/20; G06T 7/70; H04R 1/02

(52) U.S. Cl. CPC A61B 3/102; G06T 3/4038; G06T 7/0012; G06T 7/20; G06T 7/70; G06T 2207/10101; G06T 2207/30041; H04R 1/028; H04R 2499/15

(57) ABSTRACT

An apparatus, systems, and methods for a wearable device configured for performing ophthalmic imaging via optical coherence tomography (OCT) are described. The device simultaneously acquires images of both eyes of a subject while the subject is awake and actively engaged in visual content that is displayed within the device. Imaging conducted via the device does not require the implementation of two separate OCT systems for each eye, but rather parallelizes the imaging capability of a single system. The device tracks and registers the positions of the subject's eyes relative to the imaging position, thereby generating widefield images based on the distribution of the eyes'positions. These imaging techniques have application in obtaining medically useful ophthalmic images of subjects for whom it is difficult to cooperate with traditional OCT imaging methods, such as young children or patients with severe physical or cognitive disabilities.

### CROSS-REFERENCE TO RELATED APPLICATION

This application claims the benefit of priority to U.S. Provisional Application No. 63/701,849, filed Oct. 1, 2024, the content and disclosure of which are incorporated herein by reference in their entirety.

### STATEMENT REGARDING FEDERALLY SPONSORED RESEARCH AND DEVELOPMENT

This invention was made with government support under D24AC00039 awarded by the Advanced Research Projects Agency for Health. The government has certain rights in the invention.

### BACKGROUND

Optical coherence tomography (OCT) is a biomedical imaging modality capable of producing three-dimensional cross-sectional images of biological tissue at micrometer-scale resolution and millimeter-scale depth. It can acquire such images completely noninvasively, allowing biological structures to be visualized in-vivo at resolutions comparable to histopathology, but without the need to physically remove tissue. One of the most clinically important applications of OCT is in ophthalmology, where it is used as a standard diagnostic tool for detection and/or diagnosing conditions and/or diseases including but not limited to diabetic retinopathy, macular degeneration, glaucoma, and/or other retinal and corneal diseases.

Current commercial OCT systems are implemented using a single laser beam that is directed to the patient's eye by a series of lenses, where it can be two-dimensionally scanned across the retina or cornea. The existence of a single so-called “sample arm” means that each of the patient's two eyes must be imaged individually. Additionally, existing OCT systems are relatively large and require the patient to rest their head in an immobilizing structure while they position their eye in the path of the laser beam. This level of patient cooperation is difficult to effectively impossible for certain groups such as young children and/or people with cognitive disabilities, with the diagnosis of ocular diseases in these groups being equally imperative for these groups.

These drawbacks in conventional systems motivate the development of an ophthalmic OCT system that minimizes the patient's involvement in the imaging procedure, that is, a device which can image both eyes simultaneously and adapt to the subject's physical position.

This background section is intended to introduce the reader to various aspects of art that may be related to various aspects of the present disclosure described below. This discussion is believed to be helpful in providing the reader with background information to facilitate a better understanding of the various aspects of the present disclosure. Accordingly, it should be understood that these statements are to be read in this light, and not as admissions of prior art.

### BRIEF SUMMARY

One aspect of the present disclosure is an imaging system for dual site imaging. The imaging system includes a wearable device configured to be worn by a subject. The wearable device includes a set of imaging components including a light source and a sample arm including one or more lenses and one or more scanning mirrors. The imaging system further includes a computing device communicatively coupled to the wearable device. The computing device includes at least one processor and at least one memory in communication with the at least one processor. The at least one processor programmed to execute, while the subject is wearing the wearable device, a dual site image data acquisition operation configured to control the set of imaging components to simultaneously obtain (i) first image data of a first body portion of a body of the subject and (ii) second image data of a second body portion of the subject's body. The at least one processor is further programmed to execute a dual site image data processing operation on the first image data and the second image data. The at least one processor is further programmed to generate, based on an output from the dual site image data processing operation, one or more images depicting the first body portion and the second body portion.

Another aspect of the present disclosure is a dual site imaging method implemented using (i) a wearable device configured to be worn by a subject, the wearable device including a set of imaging components including a light source and a sample arm including one or more lenses and one or more scanning mirrors, and (ii) a computing device communicatively coupled to the wearable device, the computing device including at least one processor and at least one memory in communication with the at least one processor. The dual site imaging method includes providing the wearable device for wearing by the subject. The dual site imaging method includes executing, while the subject is wearing the wearable device, and by the computing device, a dual site image data acquisition operation configured to control the set of imaging components to simultaneously obtain (i) first image data of a first body portion of a body of the subject and (ii) second image data of a second body portion of the subject's body. The dual site imaging method includes executing, by the computing device, a dual site image data processing operation on the first image data and the second image data. The dual site imaging method includes generating, based on an output from the dual site image data processing operation, and by the computing device, one or more images depicting the first body portion and the second body portion.

In some embodiments, the device is also able to track and register the positions of the subject's eyes relative to the imaging position, thereby generating widefield images based on the distribution of the eyes'positions, and/or capture images of different portions of the eye. Moreover, additional sites (e.g., more than two sites) can be ascertained by the techniques described herein. For example, the techniques described herein are not limited to dual object/dual site imaging, but rather encompass multi-object/multi-site imaging.

Various refinements exist of the features noted in relation to the above-mentioned aspects. Further features may also be incorporated in the above-mentioned aspects. These refinements and additional features may exist individually or in any combination. For instance, various features discussed below in relation to any of the illustrated embodiments may be incorporated into any of the above-described aspects, alone or in any combination.

### BRIEF DESCRIPTION OF THE DRAWINGS

The following figures illustrate various aspects of the disclosure. The patent or application file contains at least one drawing executed in color. Copies of this patent or patent application publication with color drawing(s) will be provided by the Office upon request and payment of the necessary fee.

FIG. 1 is a schematic diagram illustrating a wearable dual-eye OCT system including an OCT optical circuit according to one embodiment of the present disclosure.

FIG. 2 depicts an OCT optical circuit diagram according of an alternative OCT optical circuit according to one embodiment of the present disclosure.

FIG. 3 depicts individual images of each eye appearing at different axial depths in a final processed image due to optical delay introduced in the device according to one embodiment of the present disclosure.

FIG. 4 is a schematic diagram illustrating one configuration of the dual optical paths for imaging according to one embodiment of the present disclosure.

FIG. 5 is a schematic diagram illustrating a side-view of a head-mounted OCT according to one embodiment of the present disclosure.

FIG. 6 is a schematic diagram illustrating a parallel multi-beam OCT on a chip using photonic integrated circuits according to one embodiment of the present disclosure.

FIG. 7 is a schematic diagram illustrating a subject being imaged by an OCT eyepiece while viewing an object located outside of the device, according to one embodiment of the present disclosure.

FIG. 8 depicts an optical ray tracing simulation according to one embodiment of the present disclosure.

FIG. 9 depicts a subject viewing media content while their eye is being scanned by an OCT system according to one embodiment of the present disclosure.

FIGS. 10A, 10B, and 10C depict three example medically relevant positions assumed by a subject as OCT imaging is being performed according to certain embodiments of the present disclosure.

FIG. 11 is a process flow of a process of acquiring widefield 3D OCT images of both eyes of a subject with using a built-in eye tracking system according to one embodiment of the present disclosure.

FIGS. 12A and 12B are schematic diagrams illustrating how an eye tracker ascertains pupil position and helps guide an incident beam through the pupil after a subject moves their eyes, according to one embodiment of the present disclosure.

FIG. 13 is a schematic diagram illustrating the use of dual sample arm OCT being applied to ear imaging according to one embodiment.

FIG. 14 is a schematic diagram illustrating a side-view of a head-mounted OCT device which focuses an imaging beam onto the anterior segment of a subject by way of lenses and mirrors according to one embodiment of the present disclosure.

FIG. 15 is a schematic diagram illustrating a top-view of a head-mounted OCT device which simultaneously focuses two imaging beams onto the anterior segments of both the left and right eyes of a subject by way of lenses and mirrors according to one embodiment of the present disclosure.

FIG. 16 is a schematic diagram illustrating a dual-eye wearable OCT headset connected by two optical fibers to an external OCT engine according to one embodiment of the present disclosure.

FIG. 17 depicts an optical ray tracing simulation from simulation software according to one embodiment of the present disclosure.

FIG. 18 depicts individual images of the anterior segment of each eye appearing at different axial depths in a final processed image due to optical delay introduced in the device according to one embodiment of the present disclosure.

FIG. 19 is a process flow of a process for dual anterior imaging according to one embodiment of the present disclosure.

FIGS. 20A, 20B, and 20C depict scenarios in which a gaze of a single eye is guided to different locations by way of a fixation target during OCT imaging of an eye according to one embodiment of the present disclosure.

FIGS. 21A and 21B depict scenarios in which a gaze of two eyes is guided to different locations by way of a stereoscopic fixation target pair during simultaneous dual-eye OCT imaging of an eye/eyes according to one embodiment of the present disclosure.

FIGS. 22A and 22B depict scenarios in which a gaze of two eyes is guided to different locations by way of a single target graphic display during simultaneous dual-eye OCT imaging of an eye/eyes according to one embodiment of the present disclosure.

FIG. 23 depicts a diagram showing a result of stitching together multiple independent images of different retinal locations to a single widefield image according to one embodiment of the present disclosure.

FIG. 24 is a flowchart of an OCT imaging procedure to acquire multiple independent images of retinal locations by guiding a subject's gaze, and stitching resulting images together into a single widefield image according to one embodiment of the present disclosure.

FIG. 25 depicts a block diagram schematically illustrating an example system according to one embodiment of the present disclosure.

FIG. 26 depicts an example component configuration of a computing device according to one embodiment of the present disclosure.

FIG. 27 depicts an example configuration of a remote or user computing device according to one embodiment of the present disclosure.

FIG. 28 depicts an example configuration of a server system according to one embodiment of the present disclosure.

Corresponding reference characters indicate corresponding parts throughout the drawings.

### DETAILED DESCRIPTION

Described herein are an apparatus, system, and method for a device for performing medical procedures including ophthalmic (e.g., eye) imaging, ear imaging, and/or other imaging of other portions of the body (e.g., heart, brain, skin, teeth) and/or other objects/samples, via techniques including but not limited to optical coherence tomography (OCT). The devices described herein may be wearable by a subject (e.g., a patient) and, for ophthalmic imaging, configured to simultaneously acquiring images of both eyes of the patient both while they are awake and are actively engaged in viewing visual content that is displayed by (e.g., within) the device (e.g., via a display of the device). The techniques described herein do not require the implementation of two separate OCT systems for each eye, but rather parallelizes the imaging capability of a single system, representing an improvement over conventional imaging systems. The devices described herein are also configured to track and register the positions of the patient's eyes relative to the imaging position, thereby generating wide field images based on the distribution of the eyes'positions. The techniques described herein are applicable for obtaining medically useful images including ophthalmic images of patients for whom it is difficult to cooperate with traditional OCT imaging methods, such as young children or patients with severe physical or cognitive disabilities, by way of a smaller device form factor than conventional systems.

In an OCT system, the sample arm and the reference arm are two components of the (e.g., interferometer) setup that enable imaging such as depth-resolved imaging. The sample arm directs light into the sample (e.g., tissue or object) being imaged, and sends low-coherence light to the sample. The backscattered or reflected light is collected from different depths within the sample. The amount and timing of reflected light vary depending on the internal structure of the sample, which encodes depth information. The reference arm provides a known optical path length for interference, and sends light to a mirror or reflective surface with a fixed or controllable position. The reflected light from the reference arm interferes with the light returning from the sample arm. The interference pattern between the sample and reference arms may be used to reconstruct depth profiles of the sample.

The sample arm of an OCT system is a physical pathway that delivers light to the sample and collects the backscattered signal. Its components are designed, for example, to optimize imaging resolution, depth, and/or speed. The sample arm may include an optical fiber (e.g., fiber optic cable) that transmits light from a light source (e.g., a laser beam split by a beam splitter) to the sample arm. An emitter may be placed at one end of the fiber to emit light to the various downstream imaging components. A single-mode fiber may be used for high-resolution imaging. A collimator may be used to convert diverging light from the fiber into a parallel beam, preparing it for scanning and focusing. A scanning mechanism may direct the light beam across the sample—the scanning mechanism may be galvo mirrors, micro-electromechanical system (MEMS) scanners, and/or rotating prisms or polygon mirrors. Focusing may be implemented by optics lenses to focus the light into the object/sample. The choice of lens affects lateral resolution and depth of field. Return path optics may be implemented to collect backscattered light from the sample and direct it back into the fiber for interference with the reference arm. Beam splitters and/or dichroic mirrors may be implemented in some embodiments to help separate illumination and detection paths and/or integrate additional imaging modalities.

In some embodiments, the light source (e.g., laser) in the OCT system may be located external to the system but still in operative connection with the sample arm. Other embodiments may integrate the light source into the OCT system such as part of a compact form factor (e.g., self-contained) OCT unit. The light source may be a superluminescent diode (SLD), tunable laser, or broadband source, and be positioned before the interferometer components. An output of the light source may be split by a beam splitter or fiber coupler into one or more paths that may include a path that goes to the sample arm (e.g., to illuminate the tissue or object) and a path that goes to the reference arm (e.g., to provide a known path length for interference). The light source may be configured and located in such a manner to allow the same source to serve both arms, to ensure coherence and synchronization between the sample and reference signals, and/or to simplify maintenance and/or allow for modular upgrades of the light source.

The sample and reference arms may be configured as part of an interferometer configuration, where light from a broadband source is split into two paths—one going to the sample and the other going to the reference arm. The light returning from both paths is recombined to produce interference patterns that encode depth information. For example, the OCT system may use low-coherence interferometry such that when light from both arms recombines, interference occurs only if the path lengths match within the coherence length of the light source. By scanning the reference arm or using techniques such as Fourier-domain techniques, the system can build up an image of the sample/object, such as a 2D and/or 3D image of the sample/object.

The sample arm may include lenses, mirrors, and/or a scanning mechanism (such as galvo mirrors or MEMS scanners) that direct light into the sample and collect the reflected light. The reference arm may include a mirror mounted on a translation stage or a fixed mirror, depending on whether the type of system (e.g., time-domain or Fourier-domain OCT).

Software associated with the imaging system (e.g., the OCT system) may be configured to, without limitation, control various aspects of the system, including but not limited to controlling scanning mechanisms, processing interference signals, reconstructing images from raw data, and/or managing synchronization and calibration.

### Oct Imaging Systems and Methods

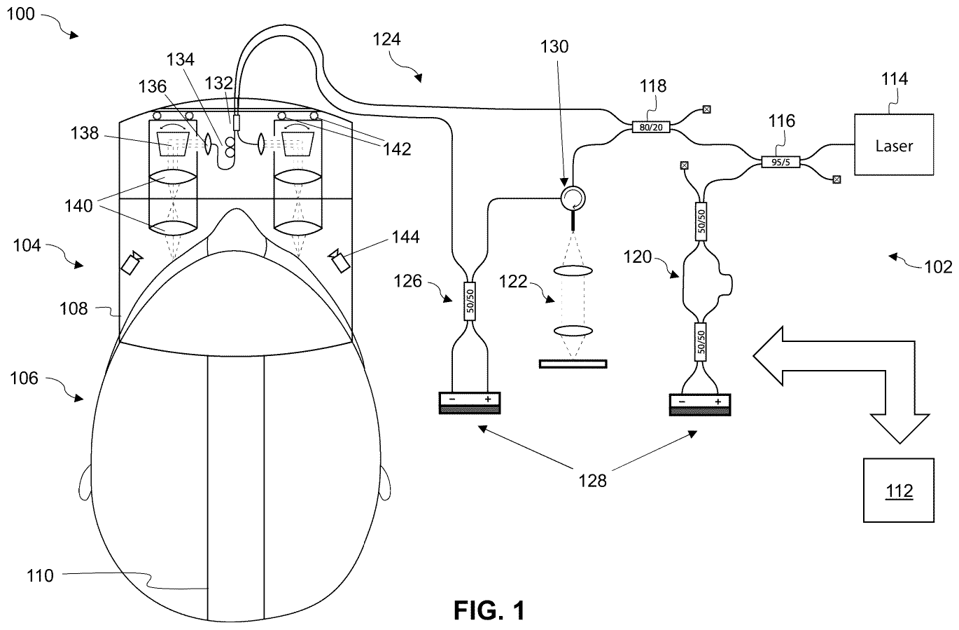

FIG. 1 is a schematic diagram illustrating one embodiment of an imaging system 100. In the embodiment shown in FIG. 1, imaging system 100 includes an OCT system 102 and an imaging device 104. OCT system 102 may be contained within imaging device 104. Imaging system 100 may include aspects and/or configurations to enable wearability. For example, a subject 106 such as a patient may wear imaging device 104 on their head (or other body part(s)). In the embodiment shown in FIG. 1, subject 106 may wear imaging device 104 on their head so that portions of the subject's body may be analyzed via OCT system 102 as described herein.

More specifically, FIG. 1 shows an example of a wearable dual-eye OCT system connected via optical fiber to a wearable eyepiece 108 that directs light from the OCT system into both eyes to achieve ophthalmic imaging. Eyepiece 108 may contain lenses, scanning mirrors, an OCT sample arm, and/or displays to facilitate the techniques of imaging as outlined herein. Eyepiece 108 may be configured as a head-mounted device able to be worn on a head of subject 106 via a securing mechanism 110 such as a strap (e.g., an adjustable strap). Eyepiece 108 may be optically connected to an external system 112 such as an external computing system that performs interferometry, data acquisition, and/or processing as described herein. Focusing on OCT system 102, FIG. 1 depicts one example of OCT system 102 that uses a wavelength-tunable light source (e.g., swept source). OCT system 102 may generally include a light source 114 such as a wide bandwidth light source (e.g., a laser such as a swept-source laser), an optical splitting device 116 such as a 95/5 optical coupler, another optical splitting device 118 such as an 80/20 optical coupler, an interferometer 120 such as Mach Zender Interferometer (MZI) used for calibration of the OCT signal, a reference arm 122, a sample arm 124 directed to the object (e.g., body portion) intended to be imaged, an optical coupler 126 such as a 50/50 optical coupler, and one or more photoelements such as a set of photodetectors 128. Back reflected light from sample arm 124 and reference arm 122 interfere at coupler 126 (e.g., a 50/50 coupler), and the interference pattern measured by a photodetector (e.g., of 128) generates an OCT image as described herein. OCT system 102 may also include one or more optical circulators 130 that have ports and are capable of directing optical power from one port to another, as described herein.

In some embodiments, light source 114 may be a wavelength tunable laser that emits light over a narrow spectral bandwidth but sweeps the center wavelength of emission over a wide bandwidth. In this scenario, the narrow instantaneous linewidth of emission yields a long coherence length, allowing for large range in the imaging depth. A commercially available swept-source laser such as SL104071 from Thorlabs Inc. may be used as light source 114, which has a sweeping rate of 400 kHz, which is directly correlated to the amount of time required to acquire one axial scan (e.g., A-scan) of the sample. This laser source sweeps across a wide bandwidth of 100 nm, allowing for desired axial resolution in the final image. For such a configuration as in FIG. 1, other tunable light sources may be used, such as a vertical cavity surface emitting laser (VCSEL), a Fourier domain mode locking (FDML) laser, or a MEMS tunable laser.

Alternatively, OCT system 102 may be implemented using a laser source emitting continuously over a wide spectral bandwidth, such as a superluminescent diode (SLD) laser. In this configuration, the photodetector (e.g., of 128) in FIG. 1 is replaced with a spectrometer capable of measuring the interference signal across a wide spectral bandwidth. Additionally, the MZI subsystem is not required as it aims to address a nonlinear phase issue common among swept-source lasers. In this configuration, optical splitting device 116 (e.g., also referred to as a 95/5 optical coupler or just as a coupler) in FIG. 1 may be omitted, along with the MZI system (e.g., 120) and the associated photodetector (e.g., of 128).

Regardless of the type of laser source used, it should have the capability of emitting light at an appropriate center wavelength for imaging such as ophthalmic imaging, and over a wide enough bandwidth to have appropriate axial resolution. To image the retina, the vitreous of the eye must preferably be transparent to the center wavelength. For example, the center wavelength may be at 600 nm, 800 nm, or 1060 nm for retinal imaging. The center wavelength may also be 1310 nm to image the anterior segment of the eye. Taking a center wavelength of 1060 nm as an example, the bandwidth, or sweeping range of such a light source may be 100 nm to achieve an axial resolution of ˜5 μm. It is understood that the appropriate bandwidth for other center wavelengths can be calculated, and the optimal light source may be selected accordingly.

As depicted in FIG. 1, coupler 116 (e.g., a 95/5 optical coupler) is configured to divert 5% of the optical power to the MZI system (e.g., 120), which passing along 95% of the power to sample arm 124 and reference arm 122. Optical splitting device 118 (e.g., also referred to as an 80/20 optical coupler or just as a coupler) then sends 80% of the remaining optical power to sample arm 124 while sending 20% to reference arm 122. It is understood that these splitting ratios or percentages may be varied depending on the desired performance of OCT system 102. In general, the MZI system (e.g., 120) requires very low optical power to provide an accurate calibration signal, meaning, for example, that only 5% of the power is appropriate. It is desired to have the highest amount of optical power delivered to the sample as increased power on the sample leads to an improved signal to noise ratio (SNR). A sufficient amount of power is delivered to reference arm 122 to achieve shot noise limited imaging.

Photodetectors 128 in FIG. 1 are depicted as dual-balanced photodetectors, which have two optical input ports and convert the optical signal to an electrical signal. These devices cancel out noise that is common to the two optical signals. The use of such a photodetector such as a photodetector 128 removes background noise in the signal eliminating the need for additional postprocessing steps. A commercially available dual-balanced photodetector may be used, such as PDB471C from Thorlabs, Inc. It is understood that this device may be replaced with a single photodetector, which would require an additional background subtraction step in image processing.

It may be desired to alter the polarization of the light traveling through OCT system 102 as certain polarization orientations may lead to suboptimal imaging results. This can be achieved by implementing one or more polarization controllers (not shown) that may access arbitrary polarizations. For example, an OCT system based on optical fiber may include manual devices that alter the polarization orientation of light in optical fiber based on stress induced birefringence. These devices may also be motorized to allow for automatic polarization control by the system software.

In some embodiments, reference arm 122 may be on a motorized track as described herein such that the length can be automatically varied by the system software as described herein. The reference arm length may determine the position of the sample within the final image or the image quality due to the phenomenon of position dependent signal to noise ratio, known as sensitivity roll-off. Therefore, the automatic adjustment of the reference arm length may be a beneficial parameter to achieve maximum image quality and a standardized image format.

As depicted in FIG. 1, OCT system 102 may include one or more optical circulators (e.g., 130) that have three ports and are capable of directing optical power from one port to the adjacent port in either the clockwise or counterclockwise directions. This can prevent excess power loss due to fiber couplers by redirecting back-reflected light in a different path than its origin. In FIG. 1, a circulator 130 is placed before reference arm 122 to redirect the light reflected from a reference mirror (shown in FIG. 1 as part of reference arm 122) to a coupler (e.g., coupler 126 (e.g., a 50/50 optical coupler)) where interference with the sample arm signal will occur. Alternatively, the reference mirror could be eliminated and replaced with a single pass reference arm design. The system shown in FIG. 1 may be susceptible to certain power loss (e.g., excess power loss) of the OCT signal, due to the implementation of a 50/50 splitter as described herein to divide the sample arm power for both eyes, which will eliminate 50% of the back-reflected sample arm signal.

FIG. 1 depicts OCT system 102 sample arm 124 connected to a wearable eyepiece 108 that implements, among other functions, a mechanism to direct the imaging beam towards both eyes of the wearer to perform OCT imaging. In this embodiment, the light is split into two paths by way of a coupler 132 such as a 50/50 optical coupler, after which a specified amount of optical delay 134 (e.g. 3 mm) is introduced to make one path longer than the other. The split light then passes through two free space optical systems that may include a collimation lens 136, a scanning mirror 138, two relay lenses 140, and/or one or more tracks 142. This optical system focuses light onto either the retina or anterior segment of each eye, allowing for simultaneous high-resolution imaging of both eyes. Collimation lens 136 may also be implemented as a liquid lens with an electronically variable focal length to allow for the accommodation of subjects with different diopters. Eyepiece 108 may also include one or more cameras 144 as described herein.

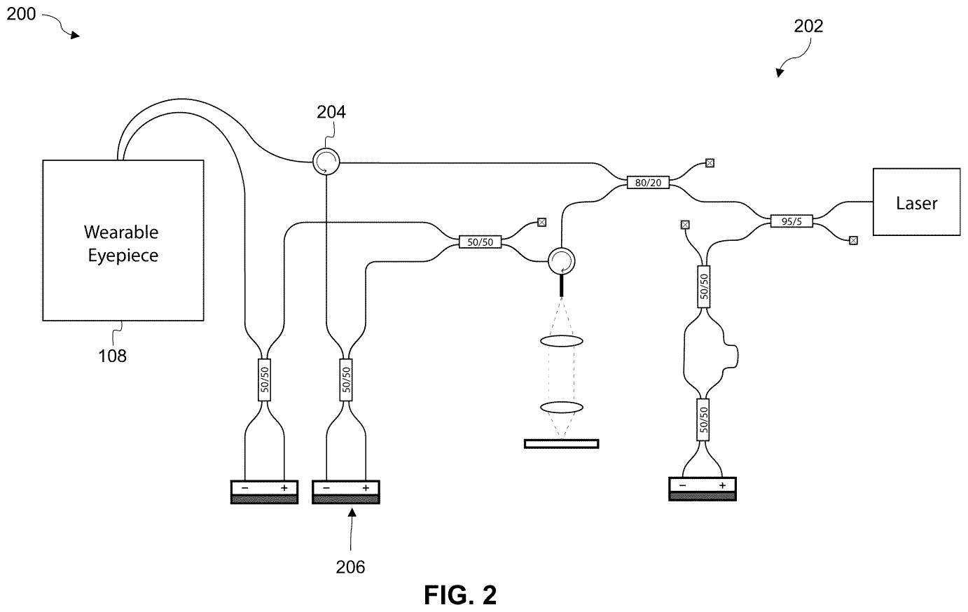

FIG. 2 depicts a diagram 200 of an OCT optical circuit 202 that is alternative to that shown in FIG. 1, which aims to recapture excess OCT signal loss to preserve signal to noise ratio. More specifically, FIG. 2 depicts an alternate OCT system setup which implements an optical circulator 204 to direct the sample arm signal travelling back through the input channel towards an additional photodetector 206 thereby conserving sample arm power and boosting the signal to noise ratio. Additionally, OCT optical circuit 202 may include one or more additional couplers (e.g., 50/50 couplers) compared to the configuration shown in FIG. 1, but may otherwise be substantially the same as or similar to the configuration shown in FIG. 1 (e.g., optical circuit 202 may include elements 114 to 130 in the configuration as shown in FIG. 1 and similarly be in operative communication with external system 112). For example, a visual comparison of the configurations shown in FIG. 1 and FIG. 2 illustrates the different (e.g., additional) components that may be present in optical circuit 202 compared to the configuration shown in FIG. 1.

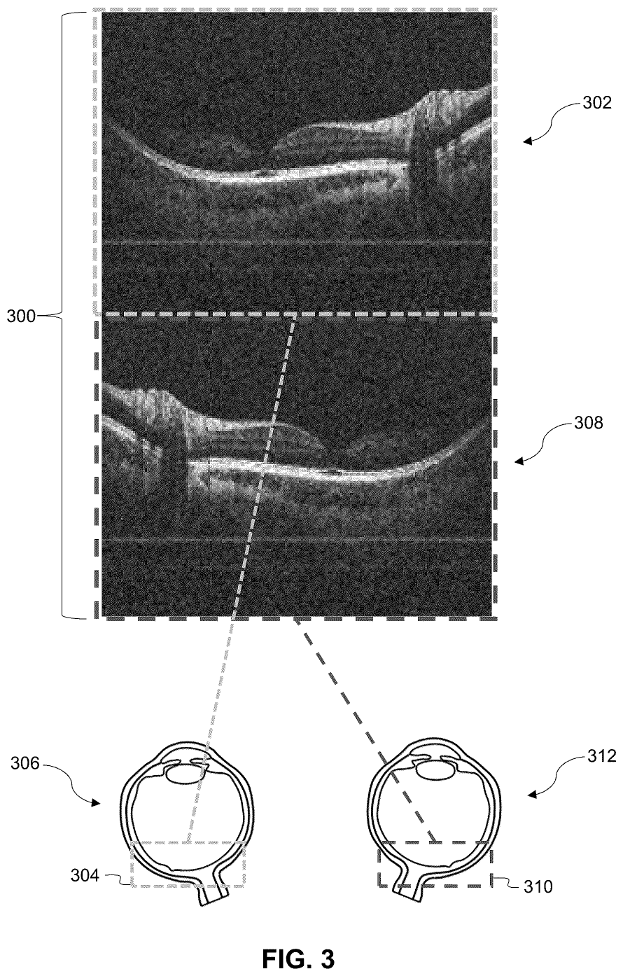

FIG. 3 depicts individual images of each eye appearing at different axial depths in a final processed image 300 due to optical delay introduced in the device according to one embodiment. Individual image 302 of final processed image 300 may show a portion 304 of one eye 306 (e.g., a left eye) of subject 106 and individual image 308 of final processed image 300 may show a portion 310 of another eye 312 (e.g., a right eye) of subject 106. Final processed image 300 and/or images 302 and 308 is/are examples of outputs from imaging system 100 resulting from the (e.g., simultaneous) imaging of dual objects (e.g., body portions), which in this example are eyes of a subject. Specifically, images 300/302/312 may be output from a device within or in communication with imaging system 100, such as external system 112 shown in FIG. 1. Portion 304 and individual image 302 are shown in yellow/dotted lines in FIG. 3. Portion 310 and individual image 308 are shown in blue/dashed lines in FIG. 3.

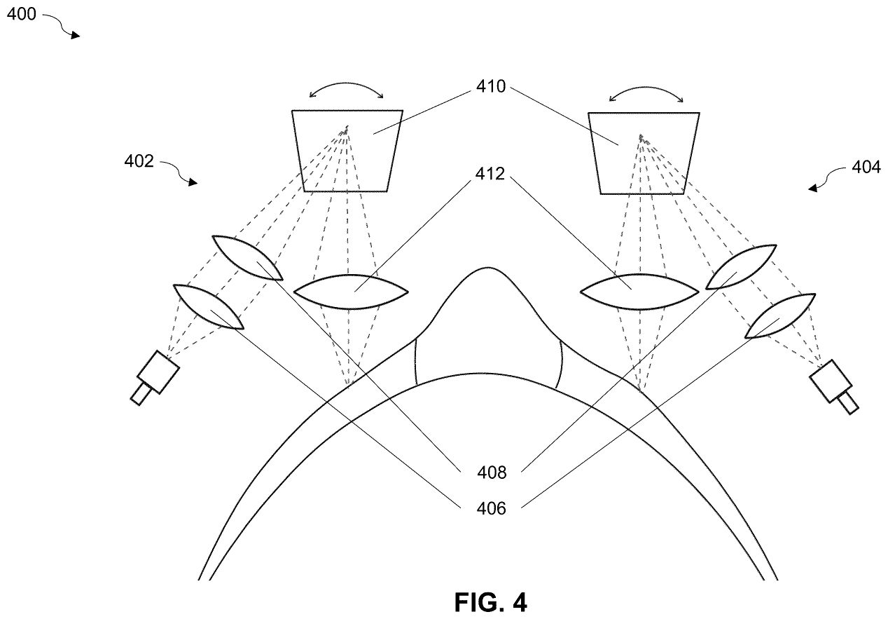

FIG. 4 is a schematic diagram illustrating one configuration of dual optical paths for imaging according to one embodiment. Diagram 400 illustrates a first optical path 402 and a second optical path 404. Components for each dual path may include first lenses 406, second lenses 408, collimation lenses 410, and third lenses 412. Dual optical paths 402 and 404 and their respective components are configured to accommodate simultaneous (e.g., dual) imaging as described herein.

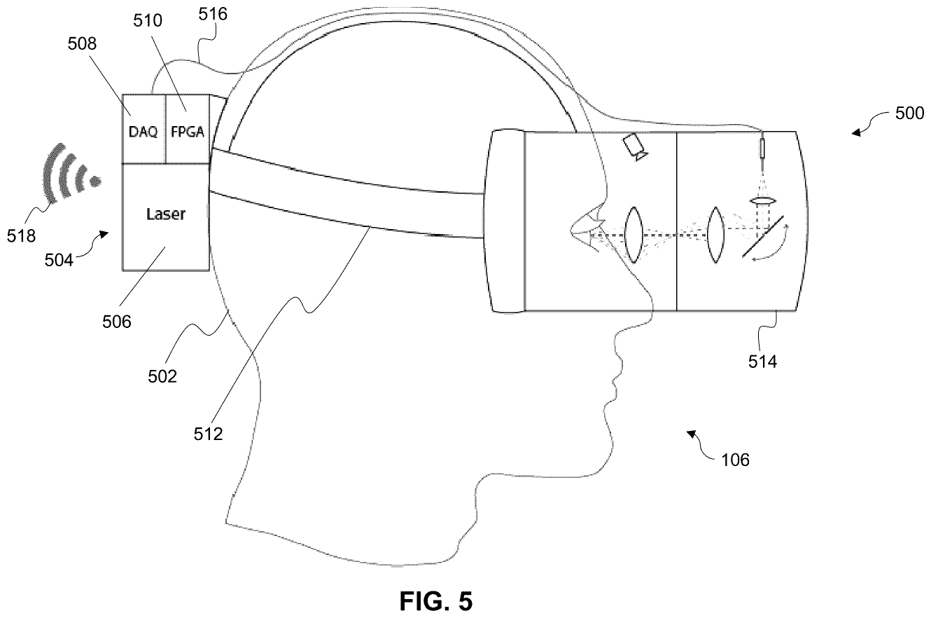

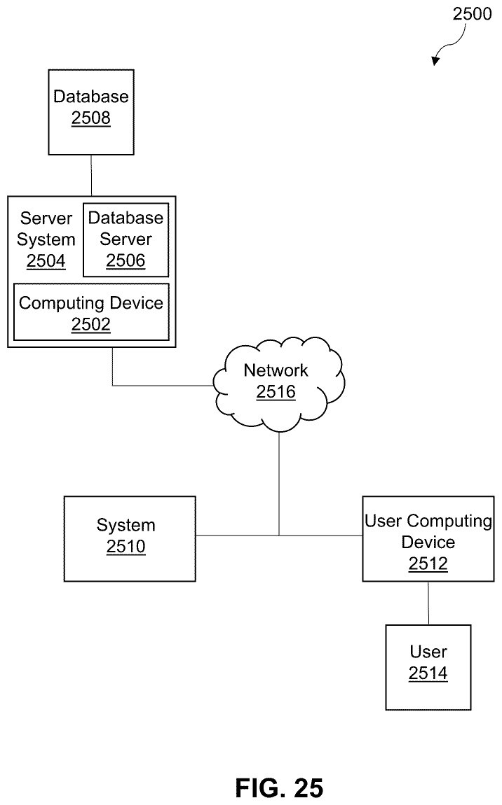

FIG. 5 depicts another configuration of wearable device 108 (shown in FIG. 1), in the form of a head-mounted OCT device 500 that includes components including but not limited to one or more lenses, one or more scanning mirrors, one or more displays, a light (e.g., laser) source, a data acquisition system (DAQ) and a field-programmable gate array (FPGA) that can wireless transmit data to a separate processing system such as external system 112 shown in FIG. 1 and/or other external systems such as one or more databases and the like. More specifically, FIG. 5 is a schematic diagram illustrating a side-view of head-mounted OCT 500 mounted to a head 502 of subject 106 and showing a modular component/unit 504 housing a laser 506, a DAQ 508, and an FPGA 510, where a head attachment mechanism such as one or more straps 512 is configured to allow for head-mounted OCT device 500 to be mounted to head 502 of subject 106. Modular component/unit 504 may be configured to attach to one or more of straps 512 so an eyepiece 514 of head-mounted OCT device 500 is a self-contained, portable unit. In some embodiments, external system 112 may, in whole or in part, be embodied as modular component/unit 504, or vice versa. A fiber optic cable 516 may be present between laser 506 and eyepiece 514 to operatively connect to each, for example so that light from laser 506 is transmitted to eyepiece 514 (e.g., to a sample arm within eyepiece 514, as described in connection with FIG. 1). Wireless transmission 518 may be configured so that data obtained by modular component/unit 504 can be wirelessly transmitted to an external system such as a database and/or other computing system, such as shown and described in connection with FIGS. 25-28. For example, in some embodiments, modular component/unit 504 may wireless transmit imaging data obtained from an imaging session to external system 112 and/or components thereof.

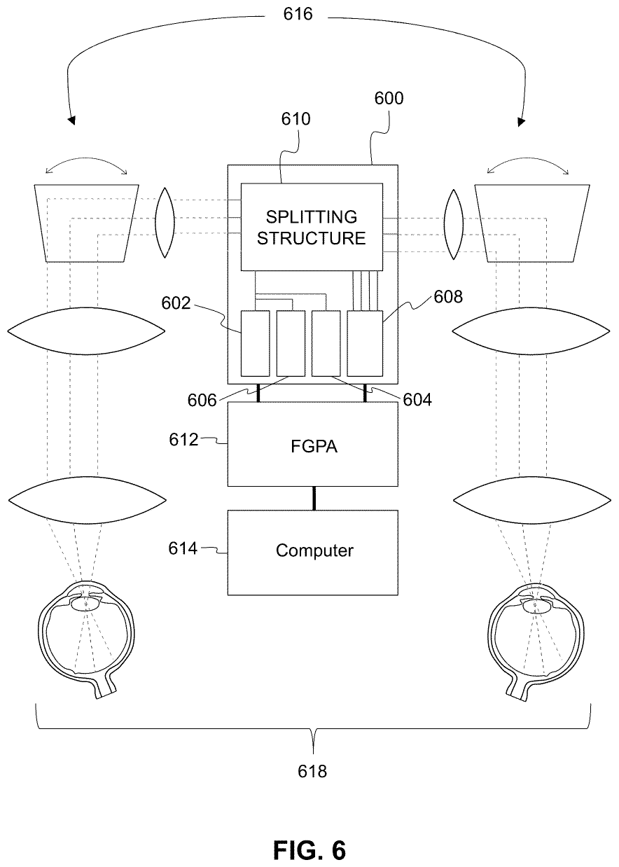

FIG. 6 is a schematic diagram illustrating an implementation of parallel multi-beam OCT on a chip 600 using photonic integrated circuits used to achieve imaging of two eyes of a subject. Chip 600 may include a laser source 602, reference arm 604, phase calibration module 606, and photodetectors 608, as well as a splitting structure 610 to parallelize the imaging beam. Chip 600 may also interface with an FPGA 612 to perform data processing and triggering, and a computer 614 to view the generated images.

More specifically, FIG. 6. depicts an alternative configuration in which an OCT engine may be implemented on chip 600, where chip 600 may be configured as a photonic integrated circuit (PIC). In this scenario, optical waveguides may be fabricated on a silicon chip by confining light inside of material with a high refractive index surrounded by a cladding material with a relatively lower refractive index. The waveguide material may be silicon (Si) or silicon nitride (Si₃N₄), lithium niobate (LN), and the cladding material may be silicon dioxide (SiO₂), among others that have the appropriate refractive index relationship. Additional active materials may be deposited onto the chip such as laser gain media (e.g. Indium phosphide, quantum dots, quantum wells, titanium sapphire), semiconductor materials (e.g., indium gallium arsenide, germanium), or electro-optic materials (e.g., lithium niobate) to facilitate the implementation of lasers, photodetectors, and tunable phase delay circuits. In this way, an OCT system can be implemented on a chip, including the splitting and optical delay for imaging both eyes simultaneously. Such function can be achieved in a smaller physical area, reducing the overall size of the device. The use of a PIC chip may allow for additional splitting of the sample arm beam to significantly increase imaging speed by way of the method of space division multiplexing (SDM). The PIC may also interface with a field programmable gate array (FPGA) to perform on-board signal processing and controlling of the OCT system. The FPGA may also interface with a computer that contains visualization software for the operator of the system. Similar to the embodiments shown in and described in connection with FIGS. 1-5, a plurality of optical elements 616 such as lenses, collimators, and the like may be configured for use with chip 600, for example to direct light to each eye of the pair of eyes 618.

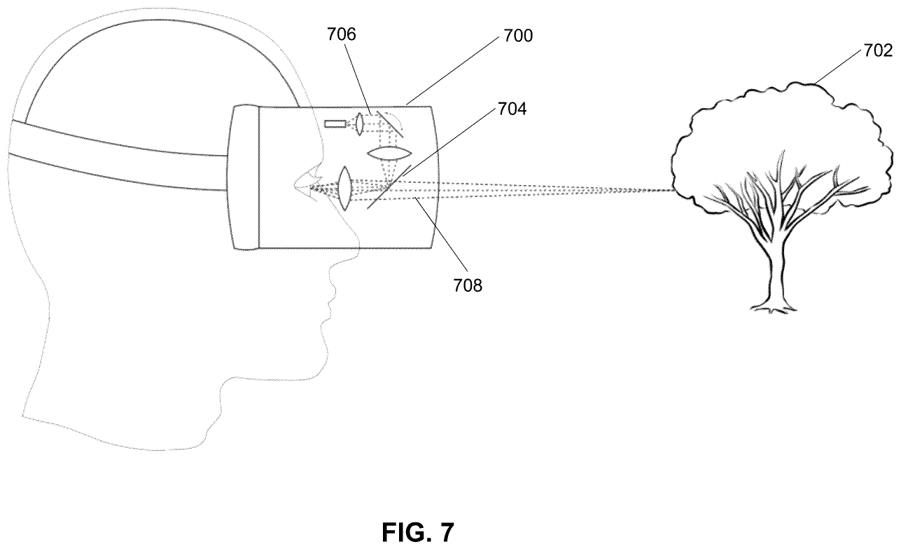

FIG. 7 is a schematic diagram illustrating a subject (e.g., patient) being imaged using another embodiment of OCT eyepiece. FIG. 7 shows a subject 106 (e.g., patient) being imaged using OCT eyepiece 700 while viewing an object located outside of the device. More specifically, FIG. 7 depicts an embodiment in which subject 106 is able to view some real object 702 in their visual field while OCT imaging of their eyes is performed via OCT eyepiece 700. For example, subject 106 may be able to watch television or read a book during the examination. This may be achieved by implementing a dichroic mirror 704 within OCT eyepiece 700 which is transparent to light in the visible spectrum but reflects the wavelength of light used for OCT imaging. This technique may be effective in configurations where the spectral bandwidth of the OCT system does not overlap with the visible spectrum, such as center wavelengths of 800 nm, 1060 nm, or 1310 nm which are in the infrared region. The red path 706 represents the OCT imaging beam, and the blue path 708 represents the patient's vision.

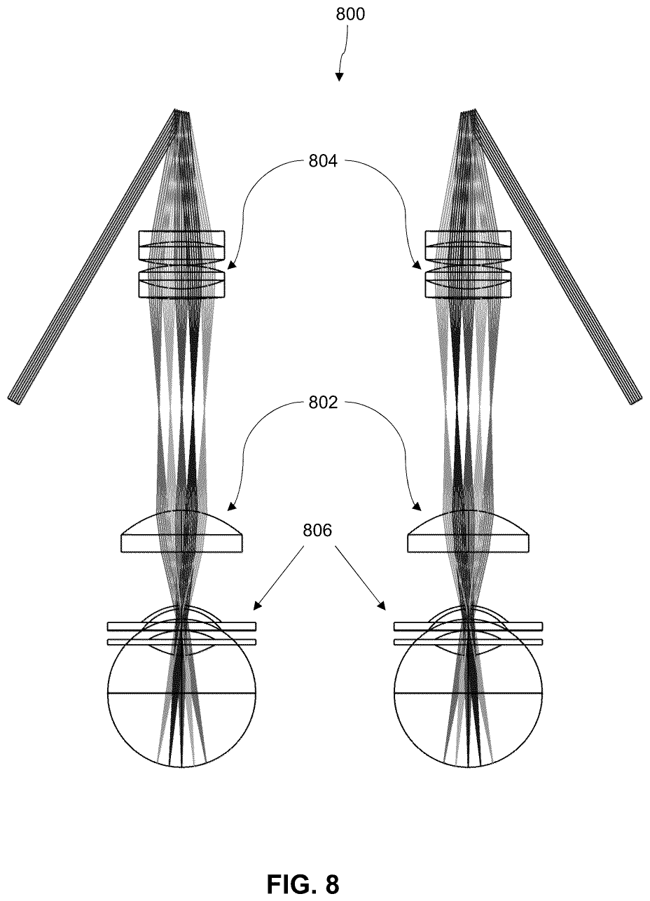

FIG. 8 depicts a result of an optical ray tracing simulation according to one embodiment. For example, simulation software (e.g., ray tracing software) such as Ansys Zemax OpticStudio that features a model of the human eye and realistically models the paths taken by light rays as they pass through various optical components may be used to conduct the simulation, and generate simulation output 800.

The simulation software models the light path through one example of a set of lenses that exhibit the desired behavior. The lenses may be achromatic doublets to correct for chromatic aberration, aspheric lenses to correct for spherical aberration, or any specialized lens type that improves the performance of the imaging system. The positions of the optical elements may be varied to achieve a more compact design of the eyepieces described herein. For example, in one embodiment, collimation lens 802 and first relay lens 804 are folded before the light is projected onto a scanning mirror to reduce the overall size required of the eyepiece. Elements 806 reflect parts of the eye model from the simulation (e.g., displayed as rectangles). In general, the space in between lenses may be folded in any way as long as a mirror is included to direct the light along the axial path.

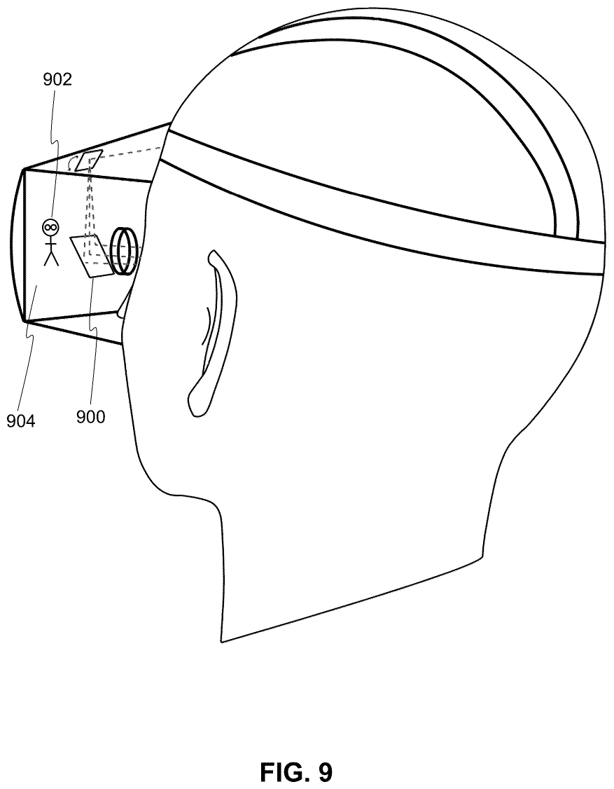

FIG. 9 depicts a similar embodiment as FIG. 7 in which a dichroic mirror 900 is used to reflect and direct the OCT imaging beam into a subject's eye, while they are able to view media content 902 on a display screen 904 located behind dichroic mirror 900. For reference, FIGS. 1 and 4 depict other embodiments in which media content 902 is able to be projected onto a dichroic surface or display screen that is transparent to infrared light, but reflective to visible light. It is understood that a dichroic mirror reflective of or transparent to any wavelengths may be used depending on the imaging wavelength used and the desired physical layout of the device.

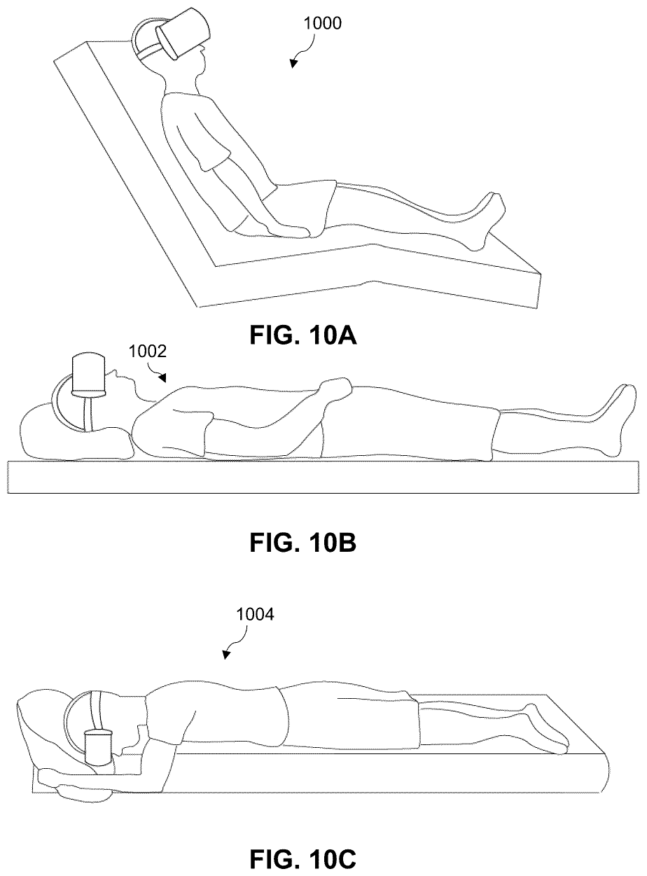

FIGS. 10A, 10B, and 10C depict three scenarios in which the eyepieces described herein and shown in the figures would be of benefit relative to conventional systems. In one scenario shown in FIG. 10A, a patient (e.g., subject 106) is sitting in a Fowler's position 1000 wearing an eyepiece of an imaging system as described herein and while their eyes are being imaged. In another scenario shown in FIG. 10B, the patient may be lying in a supine position 1002 while their eyes are being imaged by an eyepiece of an imaging system described herein. In another scenario shown in FIG. 10C, the patient may be lying in a prone position 1004 while their eyes are being imaged by an eyepiece of an imaging system described herein, which is particularly useful for patients undergoing treatment for retinal detachment, in which a vitrectomy may be performed while the patient is in the depicted position. These positions would not be able to be realized with a traditional OCT system due at least in part to the compact form factor of the wearable imaging devices described herein and/or the simultaneous imaging techniques as described herein.

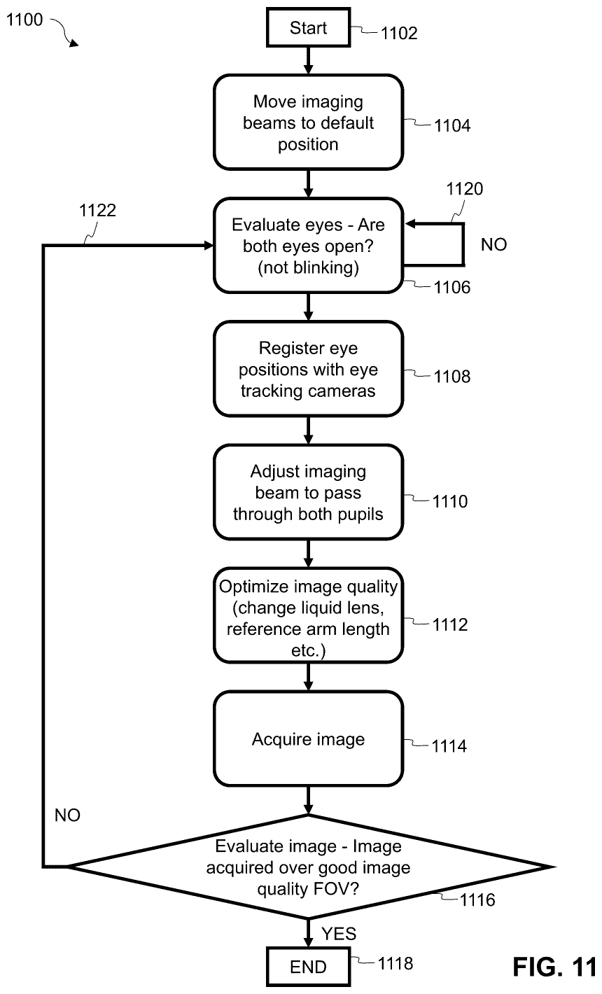

FIG. 11 depicts a flowchart for a process flow (e.g., a software control loop, specifically an image acquisition control loop) for an imaging session, including the processing of eye position information from an eye tracking system for aligning an imaging beam, and controlling hardware in an OCT system to optimize image quality according to one embodiment.

Method 1100 includes starting 1102 the process (e.g., starting a software control loop). Method 1100 further includes moving 1104 the imaging beam(s) to a default position. Method 1100 further includes evaluating 1106 a state of the subject's eye(s), such as determining if both eyes are open (e.g., not blinking). Method 1100 further includes registering 1108 eye positions with eye tracking cameras. Method 1100 further includes adjusting 1110 imaging beams to pass through both pupils. Method 1100 further includes optimizing 1112 image quality, such as by implementing various changes (e.g., changing a liquid lens, changing a reference arm length, etc.). Method 1100 further includes acquiring 1114 an image such as via the acquisition techniques described herein. Method 1100 further includes evaluating 1116 the image to determine, for example, if the acquired image includes a good image quality field of view (FOV). Method 1100 further includes ending 1118 the process. Method 1100 may further include one or more feedback loops at certain junctures in the process flow. For example, method 1100 may further include a feedback loop 1120 in connection with evaluating 1106 a state of the subject's eye(s). If the eyes are determined to be not open (e.g., “NO” at evaluating 1106), the process flow may not process to registering 1108 until a determination is made that the eyes are open. If “YES” at evaluating 1106, the process flow simply proceeds to registering 1108. Method 1100 may also include feedback loop 1122 in connection with evaluating 1116 the image to determine if the acquired image was acquired over a desired FOV. If the FOV is determined to be poor image quality (e.g., not of good image quality, as represented by “NO” at evaluating 1116), the process flow may revert to a prior point in the process flow, such as evaluating 1106 a state of the subject's eye(s). If “YES” at evaluating 1116, the process flow simply proceeds to ending 1118. Operations 1106-1116, alone or in combination, may be referred to as a dual site image data processing operation. In other embodiments, operations similar to 1106-1116, alone or in combination, may be referred to as a multi-site image data processing operation, such as when the regions of interest encompass more than two objects.

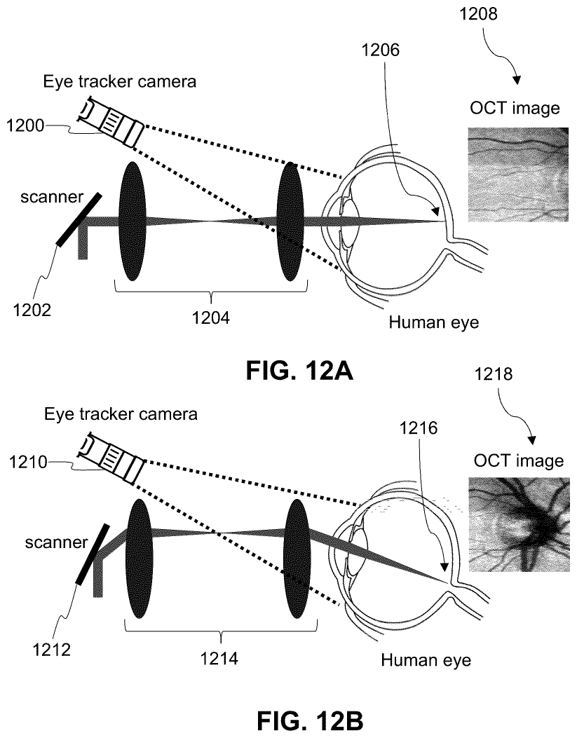

FIGS. 12A and 12B are diagrams illustrating how an eye tracker (e.g., an eye tracker camera) ascertains pupil position and helps guide an incident beam through the pupil after a subject move their eye(s), according to one embodiment. FIG. 12A shows a combination of imaging elements including an eye tracker camera 1200, a scanner 1202, and one or more lenses 1204. The combination is configured to track an eye during an imaging session where light is directed to a particular portion 1206 of the eye and/or where the eye is in a first position. The result of the imaging may include an OCT image 1208, showing portions of the eye such as portion 1206. FIG. 12B shows a combination of imaging elements including an eye tracker camera 1210, a scanner 1212, and one or more lenses 1214. The combination is configured to track an eye during an imaging session where light is directed to a particular portion 1216 of the eye and/or where the eye is in a second position that may be different than the first position. The result of the imaging may include an OCT image 1218, showing portions of the eye such as portion 1216. Scanner 1202 and scanner 1212 may be oriented at different angles to change the direction of light relative to lenses 1204 and 1214, respectively.

With reference to FIGS. 11, 12A, and 12B, first the imaging system will start up and bring the imaging beams to their default positions. Then, the image acquisition control loop will begin. The imaging system will determine whether the eyes are open or not, in the scenario that the subject may be blinking. If the eyes are closed, all imaging functions are paused until it is detected that the eyes are open. If it is determined that the eyes are open, the locations of the pupils via eye tracking cameras 1200 (FIGS. 12A) and 1208 (FIG. 12B) are established and registration of their position to the imaging beam coordinates occurs. Then, the imaging system will calculate other parameters such as an alignment error between a current imaging beam position and a position at which the beam will overlap with the pupil. The imaging system will then move the imaging beam by adjusting the lateral positions of the beams by way of a motorized mechanism such as tracks 142 shown in FIG. 1, and by adjusting the beams'angular positions by providing a biased offset to the associated scanning mirrors. This process of adjusting the imaging beam position to accommodate the position of the eye is depicted in FIGS. 12A and 12B. The imaging system will then adjust various hardware and software parameters to maximize the image quality before acquiring the image. For example, the imaging system may adjust the length of a (e.g., motorized) reference arm to place the retinal image within an appropriate position within the imaging range, change the focal length of a liquid lens, adjust the polarization of the incident light, improve the sharpness of the image by applying a numerical dispersion compensation algorithm, and/or adjusting the brightness and contrast of the image. Finally, the imaging system will initiate acquisition of the image for a preset time duration before cycling through the loop again. If the full dataset has been acquired over the desired field of view, the loop will terminate, otherwise it will repeat the loop beginning from determining whether the eye is open. The imaging system may also be configured to determine if a specific location on the retina has been missed, in which case the imaging system will cause a return to that position to complete the full dataset.

Adaptations may be made to the imaging systems described herein may be made to facilitate imaging of other portions of the body, where such imaging systems may include an imaging device configured to facilitate imaging of other body parts, such as ears, rows of teeth, skins, etc., to simultaneously capture images from a plurality of locations according to the techniques described herein.

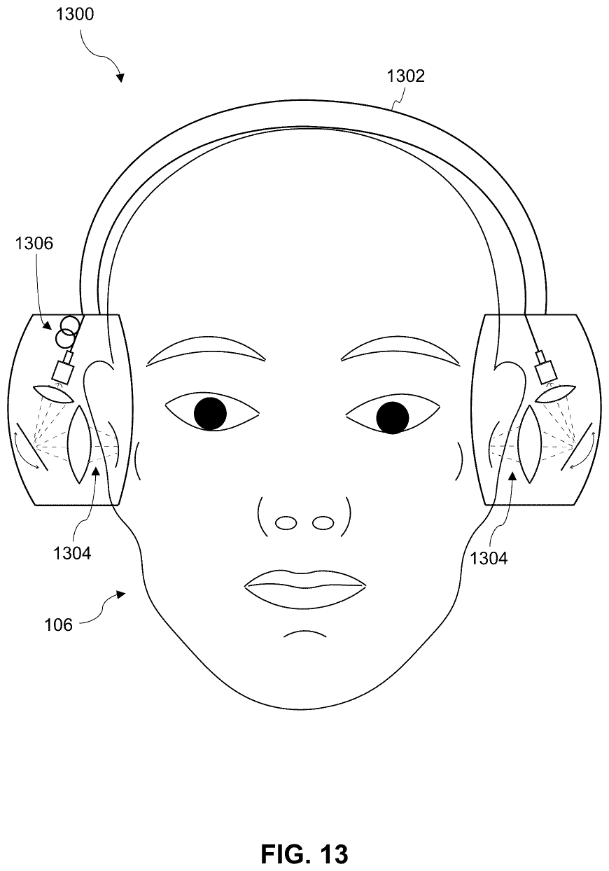

FIG. 13 depicts a dual-ear imaging device 1300 in which a subject (e.g., subject 106) wears a headset 1302 that directs an OCT imaging beam 1304 into both ears. The components on one side of headset 1302 may include optical delay 1306. By including (e.g., additional) optical delay 1306 on one side, the image of one ear will appear at a different axial depth in the final image compared to the other ear, allowing simultaneous acquisition with a single OCT system, representing a significant improvement over conventional systems. Such dual-ear imaging is beneficial for evaluating ear conditions including but not limited to middle ear infections. While there are some commonalities of imaging both eyes and ears, there are also distinct features and functionalities. For example, headset 1302 may include similar configurations of lenses, mirrors, and the like such as shown in the figures in connection with eye imaging. However, whereas for eye imaging (see FIGS. 1 and 3-12), eye tracking and displaying real scenes and media (e.g., entertainment) content may be implemented to keep the subject engaged, for ear imaging, the dual-ear imaging device may be configured to play music or other sounds via one or more speakers (not shown) to provide stimulation, for example to evaluate hearing/ear drum functions.

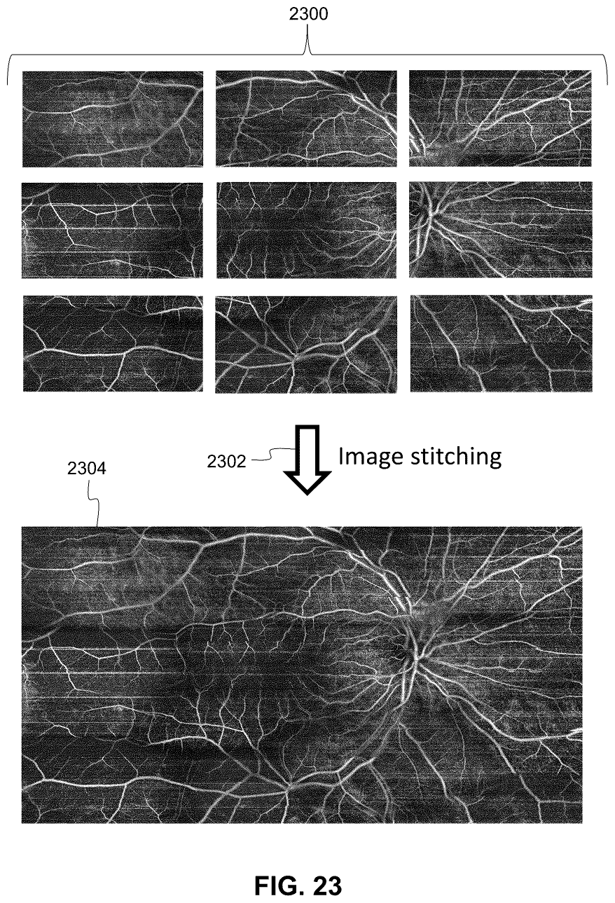

After the image acquisition session for either eyes or ears is complete, the data obtained from the session may be analyzed by post-processing software to determine, in the case of eye imaging, overlap of different images acquired from different eye positions, and, for the case of ear imaging different aspects of the ear (e.g., ear drum movement). The software may then perform stitching of the images to form a contiguous (e.g., widefield) image (such as shown in FIG. 23, described in more detail below). Image stitching may be performed to generate a contiguous (e.g., widefield) image of the retina for eye imaging, or a contiguous (e.g., widefield) image of portions of the ear (e.g., ear drum) for ear imaging.

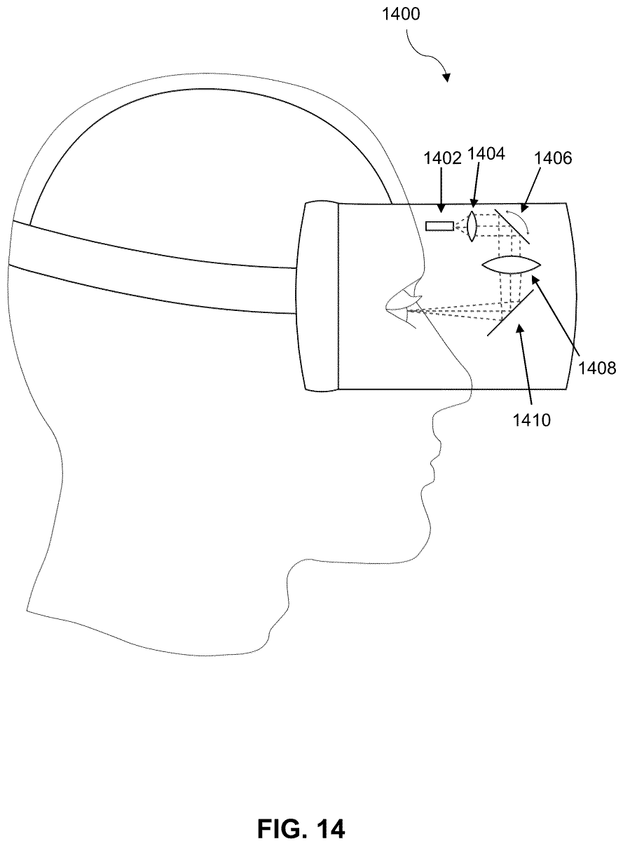

FIGS. 14-19 illustrate aspects of another eye imaging embodiment, specifically a dual anterior imaging embodiment.

FIG. 14 is a schematic diagram illustrating a side-view of a head-mounted OCT device 1400 which focuses an imaging beam onto the anterior segment of a subjects'eyes user by way imaging components including lenses and mirrors similar to other embodiments. For example, head-mounted OCT device 1400 may include an emitter 1402, a first lens 1406, a collimator 1408, a second lens 1410, and a mirror 1412.

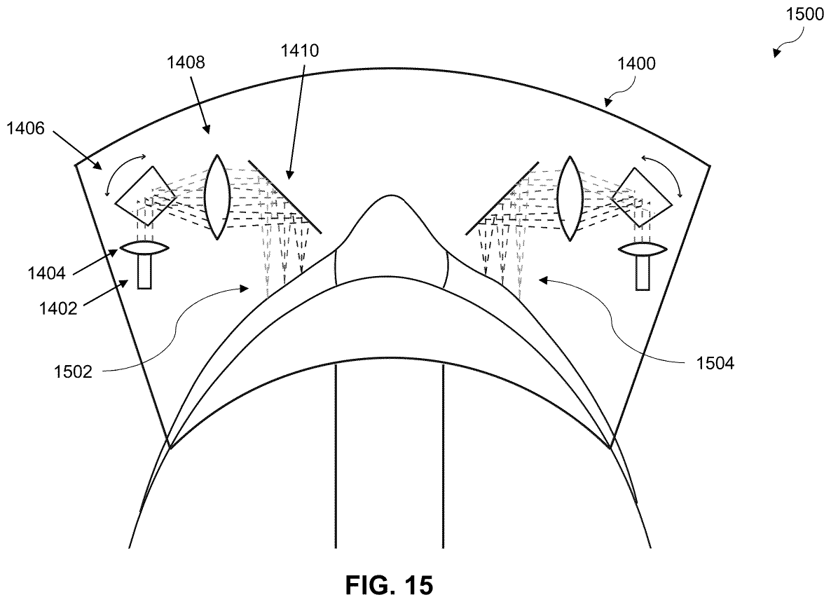

FIG. 15 is a schematic diagram illustrating a top-view 1500 of head-mounted OCT device 1400 shown in FIG. 14. As described in connection with FIG. 14, the imaging components of head-mounted OCT device 1400 are configured to simultaneously focus two imaging beams 1502 and 1504 onto the anterior segments of both the left and right eyes of the user by ways of lenses (e.g., 1406, 1410) and one or more mirrors (e.g., 1412). For reference, a comparison between the component configuration shown in FIG. 15 and that shown in FIG. 4 illustrates the differences in component arrangement relative to the dual anterior embodiment.

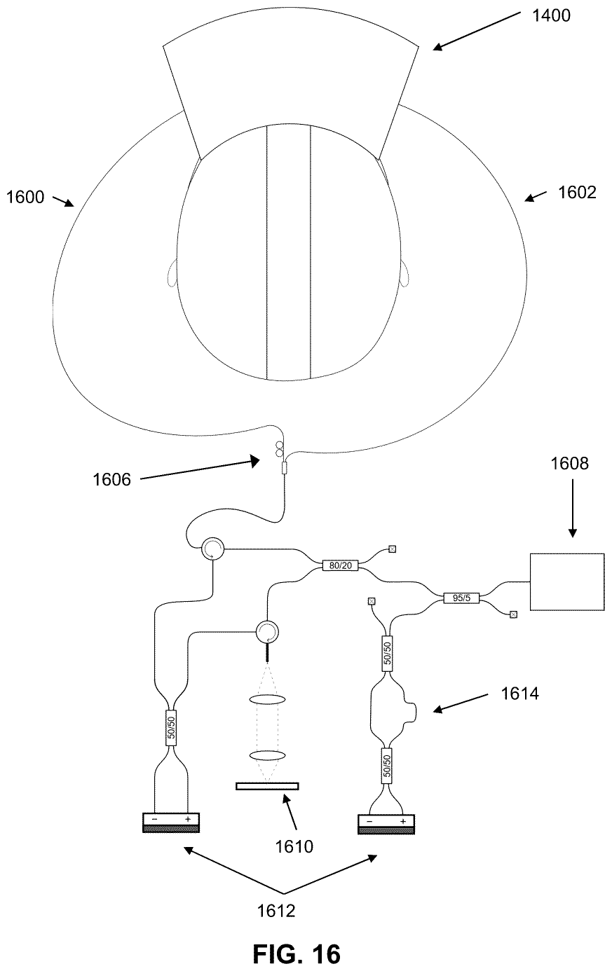

FIG. 16 is a schematic diagram illustrating another top-view of dual-eye, wearable, head-mounted OCT device 1400 connected by two optical fibers to an external OCT engine. Head-mounted OCT device 1400 includes a first optical fiber 1600 and a second optical fiber 1602, where first optical fiber 1600 may include an optical delay 1604. External OCT engine may include an OCT configuration 1606 similar to that shown in FIG. 1, including, for example, a laser 1608, a reference arm 1610, a set of photodetectors 1612, and an interferometer 1614.

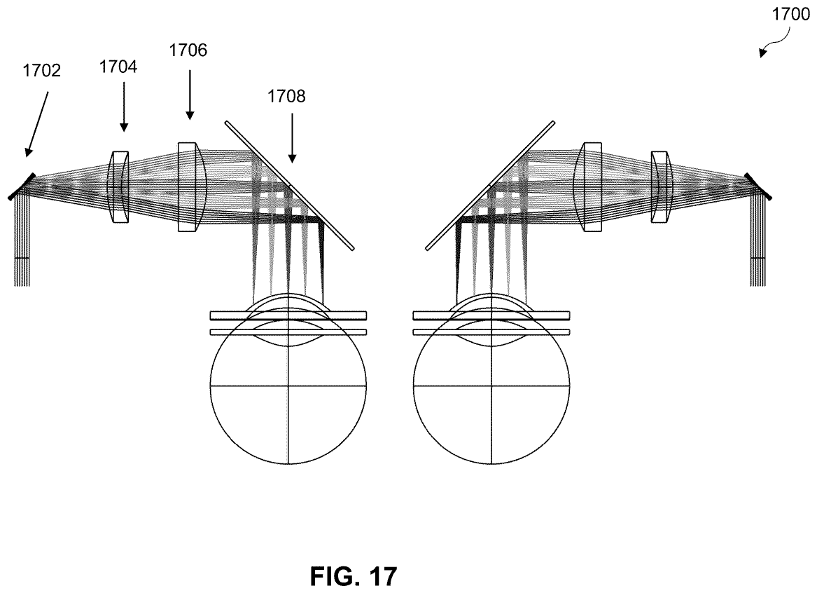

FIG. 17 depicts an optical ray tracing simulation result from simulation software. As described herein, the simulation software may be commercially available software such as Ansys Zemax OpticStudio that features a model of the human eye and realistically models the paths taken by light rays as they pass through various optical components. The simulation may provide for the placement of various imaging components in a specified order to simulate certain configurations of components. Optical ray tracing simulation output 1700 shows modeling of the light path through an example of a set of components that exhibit the desired behavior, in this case dual anterior focusing. For the simulation shown in FIG. 17, the imaging components may include, for each eye, a collimator 1702, a first lens 1704, a second lens 1706, and a mirror 1708. The lenses may be any specialized lens type that improves the performance of the imaging system.

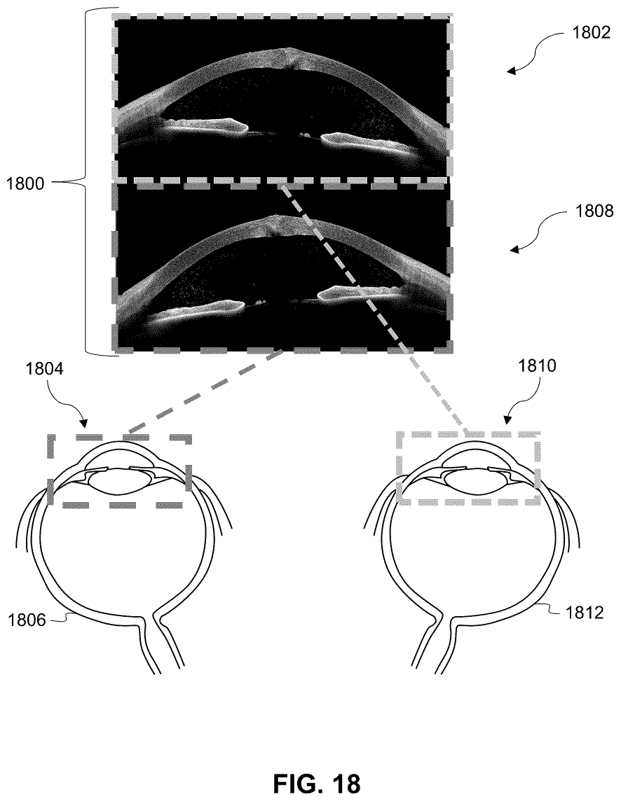

FIG. 18 depicts individual images of the anterior segment of each eye appearing at different axial depths in a final processed image due to optical delay introduced in the device for the dual anterior embodiment described herein.

Similar to FIG. 3, FIG. 18 depicts individual images of each eye appearing at different axial depths in a final processed image 1800 due to optical delay introduced in the device according to one embodiment. Individual image 1802 of final processed image 1800 may show a portion 1804 (e.g., anterior segment) of one eye 1806 (e.g., a left eye) of a subject (e.g., subject 106) and individual image 1808 of final processed image 1800 may show a portion 1810 (e.g., anterior segment) of another eye 1812 (e.g., a right eye) of subject 106. Final processed image 1800 and/or images 1802 and 1808 is/are examples of outputs from the imaging system shown in FIG. 16 resulting from the (e.g., simultaneous) imaging of dual objects (e.g., body portions), which in this example are eyes of a subject. Specifically, images 1800/1802/1812 may be output from a device within or in communication with the imaging system shown in FIG. 16, such as external system 112 shown in FIG. 1. Portion 1804 and individual image 1802 are shown in yellow/dotted lines in FIG. 18. Portion 1810 and individual image 1808 are shown in blue/dashed lines in FIG. 18.

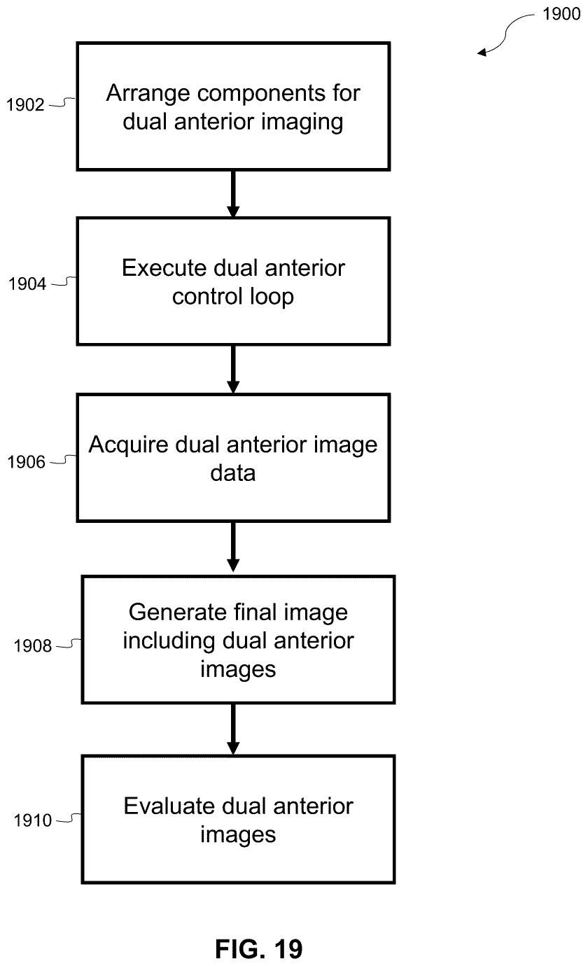

FIG. 19 is a flowchart of an OCT imaging dual anterior procedure as reflected in FIGS. 14-18. In this regard, method 1900 includes arranging 1902 imaging components for a dual anterior imaging procedure such as shown in FIG. 15. Method 1900 further includes executing 1904 a dual anterior control loop. Method 1900 further includes acquiring 1906 dual anterior image data. Method 1900 further includes generating 1908 a final image including a dual anterior image for each eye such as shown in FIG. 18. Method 1900 further includes evaluating 1910 the images.

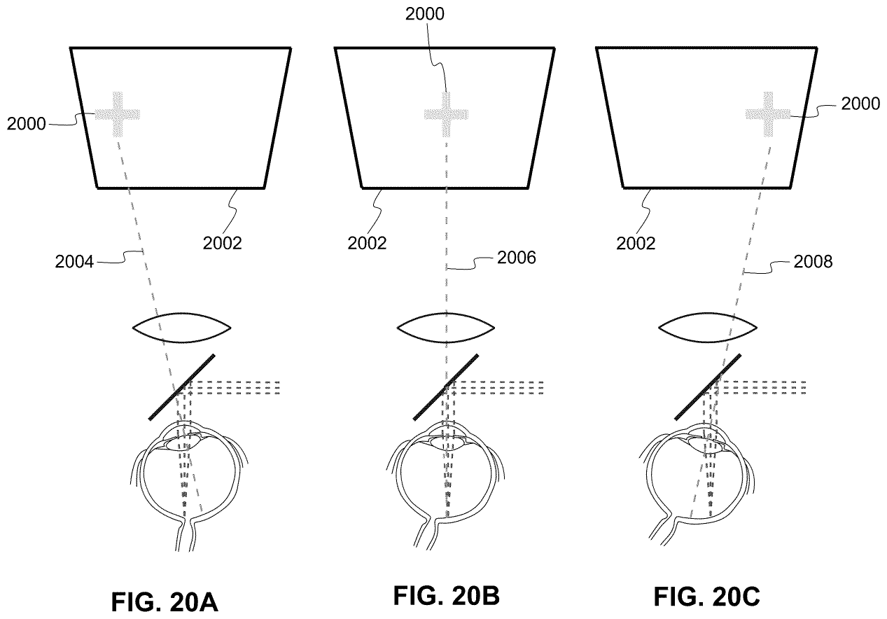

FIGS. 20-24 illustrate aspects of another eye imaging embodiment, specifically an ultrawide imaging embodiment.

FIGS. 20A to 20C depict scenarios in which a gaze of a single eye is guided to different locations by way of a fixation target during OCT imaging of the eye (e.g., of the retina). More specifically, FIGS. 20A to 20C show a fixation target 2000 in three different locations on a display 2002. As shown in FIG. 20A, fixation target 2000 may be located to guide a subject's gaze in a leftward location for imaging of the eye in a position corresponding to a leftward gaze 2004. As shown in FIG. 20B, fixation target 2000 may be located to guide a subject's gaze in a center location for imaging of the eye in a position corresponding to a centered gaze 2006. As shown in FIG. 20C, fixation target 2000 may be located to guide a subject's gaze in a rightward location for imaging of the eye in a rightward gaze 2008. Display 2002 may be a display screen that may be configured as part of a wearable eyepiece as shown and described herein, such as in connection with FIG. 9. Imaging can therefore be performed for each location and corresponding positions/gazes of the eye(s).

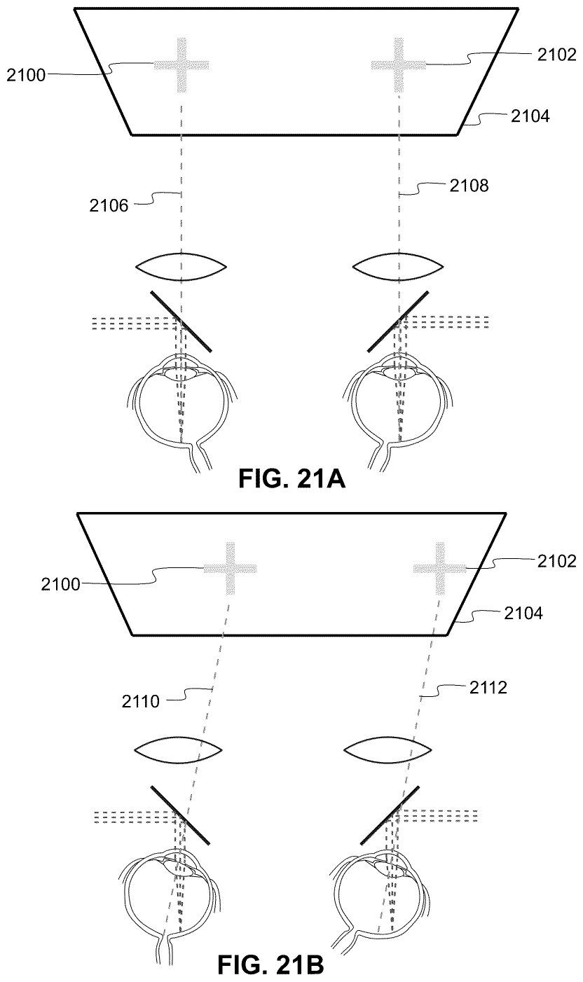

FIGS. 21A and 21B depict scenarios in which a gaze of two eyes is guided to different locations by way of a stereoscopic fixation target pair during simultaneous dual-eye OCT imaging of the eye(s) (e.g., of the retina). More specifically, FIG. 21A shows two fixation targets 2100 and 2102 aligned with the eyes of the subject on a display 2104 to guide a subject's eyes in a straight gaze, and FIG. 21B shows the two fixation targets 2100 and 2102 rightward-aligned on display 2104 to guide a subject's eyes to a rightward gaze. As shown in FIG. 21A, fixation targets 2100 and 2102 may be aligned relative to a display area of display 2104 to guide a subject's gaze for each eye in a straight location for imaging of each eye in a straight gaze, such as straight gaze 2106 for the left eye and straight gaze 2108 for the right eye. As shown in FIG. 21B, fixation targets 2100 and 2102 may be right-aligned relative to a display area of display 2104 to guide a subject's gaze for each eye in a rightward location for imaging of each eye in a rightward gaze, such as rightward gaze 2110 for the left eye and rightward gaze 2112 for the right eye. Fixation targets 2100/2102 may be treated as a pair and be displayed on display 2104. Display 2104 may be a display screen that may be configured as part of a wearable eyepiece as shown and described herein, such as in connection with FIG. 9. Imaging can therefore be performed for the location of the fixation target pair and corresponding positions/gazes of the eyes.

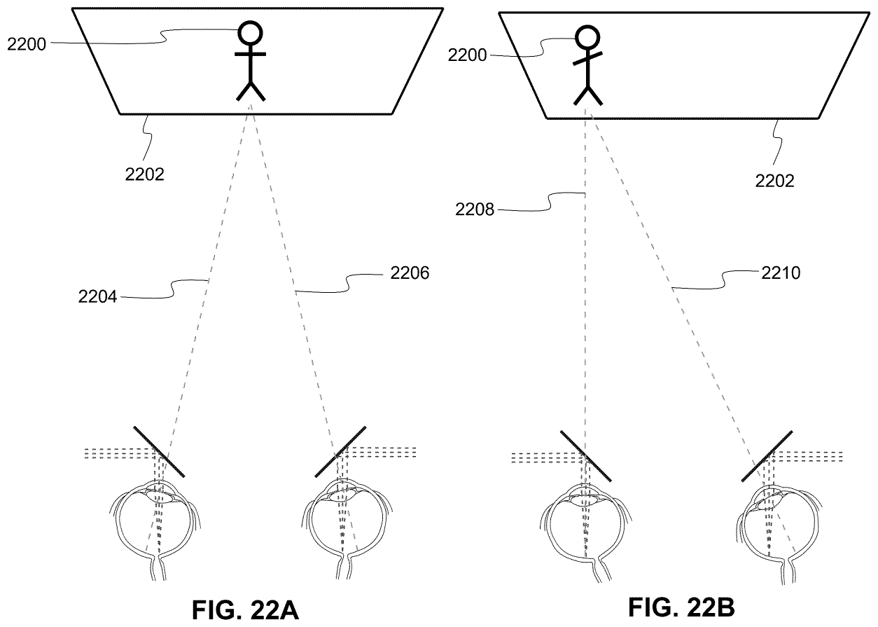

FIGS. 22A and 22B depict scenarios in which a gaze of two eyes is guided to different locations by way of a single target graphic displayed during simultaneous dual-eye OCT imaging of the eye(s) (e.g., of the retina). More specifically, FIG. 22A shows a single target graphic 2200 center-aligned on a display 2202 to guide a subject's eyes to gaze at centered single target graphic 2200, and FIG. 22B shows single target graphic 2200 leftward-aligned on display 2202 to guide a subject's eyes to gaze at leftward single target graphic 2200. As shown in FIG. 22A, single target graphic 2200 may be centered relative to a display area of display 2202 to guide a subject's gaze for each eye to the centered location for imaging of each eye in a gaze aimed at single target graphic 2200, such as angled gaze 2204 for the left eye and angled gaze 2206 for the right eye. Angled gazes 2204 and 2206 aim the eyes on centered single target graphic 2200. As shown in FIG. 22B, single target graphic 2200 may be left-aligned relative to a display area of display 2202 to guide a subject's gaze for each eye to the leftward location for imaging of each eye in a respective gaze. For example, because single target graphic 2200 is left-aligned on display 2202, one eye (e.g., the left eye) may have a straight gaze 2208, whereas the other eye (e.g., the right eye) may have a leftward gaze 2210. Display 2202 may be a display screen that may be configured as part of a wearable eyepiece as shown and described herein, such as in connection with FIG. 9. Imaging can therefore be performed for the location of single target graphic 2200 and corresponding positions/gazes of the eyes. Single target graphic 2200 may be part of displayed media content as described herein.

The targets and/or graphics shown in FIGS. 20A to 22B are merely examples and other locations may be implemented to image the eyes in other gazes/positions. For example, the targets/graphics may be top and/or bottom aligned to image eyes in corresponding (e.g., raised/lowered) positions/gazes. Additionally, the targets/graphics may have motion to track eye movement corresponding to the movement of the targets/graphics.

FIG. 23 shows a result of stitching together multiple independent images of different eye (e.g., retinal) locations to a single widefield image. For example, during an imaging session according to the widefield embodiment shown and described in connection with FIGS. 20A to 22B, a plurality of individual images 2300 of different portions of the eye may be obtained. In order to generate a single (e.g., widefield) image showing the entirety of the overall portion of the eye that was captured, individual images 2300 may be stitched together via an image stitching process 2302. A result of image stitching process 2302 is a unified single (e.g., final) widefield image 2304, representing a combination of individual images 2300. Image stitching process 2302 may include image processing software configured to take input images and generate a composite image that is an effective equivalent to as if a single image was originally captured. Image stitching process 2302 may utilize one or more advanced image processing tools such as artificial intelligence/machine learning models trained for such image processing techniques. For example, a computer vision model may be used to stitch individual images 2300 together in an appropriate order to generate final image 2304. Image stitching process 2302 may be configured to take into account aspects of the individual images such as edges, borders, overlap, and/or other aspects of individual images 2300 so that a seamless final image 2304 results. For example, a goal of image stitching process 2302 may be to output final image 2304 such that final image 2304 is indistinguishable compared to if final image 2304 was obtained via a single capture as opposed to multiple captures for each of individual images 2300. Final image 2304 may therefore represent a widefield view of the portion of the eye that was the region of interest for the imaging session, where the overall portion displayed in image 2304 is derived from the individual portions captured in each individual image 2300.

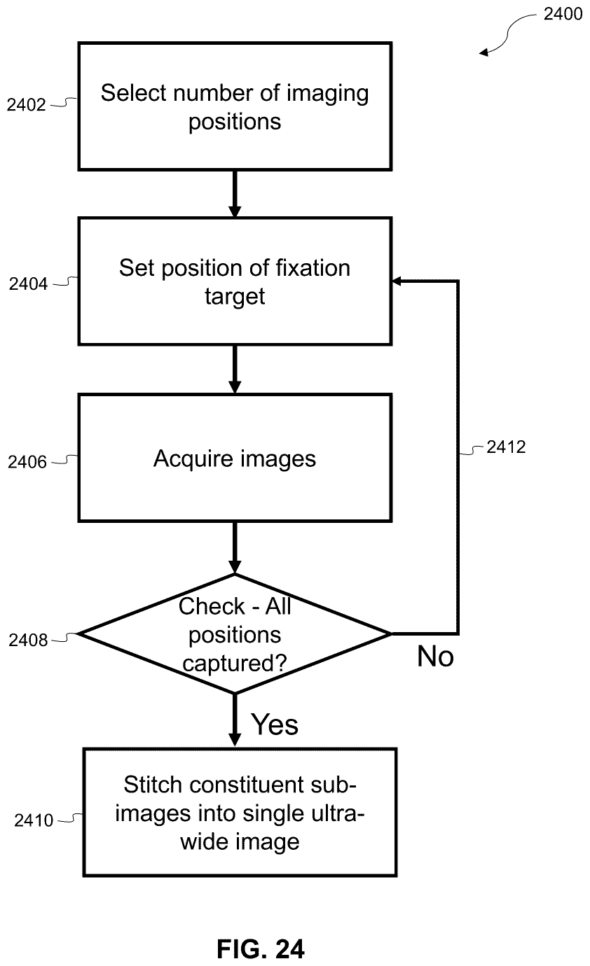

FIG. 24 is a flowchart of an OCT imaging procedure to acquire multiple independent images of retinal locations by guiding a subject's gaze, and stitching the images together into a single widefield image. In this regard, method 2400 includes selecting 2402 a number of imaging positions. This may include a numbers of positions sufficient to capture a region of interest of the eye. Method 2400 further includes setting 2404 a position of a fixation target(s)/graphic(s), such as shown and described in connection with FIGS. 20A to 22B. Method 2400 further includes acquiring 2406 images (e.g., retinal images) corresponding to the selected positions. Method 2400 further includes checking 2408 to ensure all selected positions have been captured. Method 2400 further includes stitching 2410 the various constituent sub-images (e.g., individual images 2300 shown in FIG. 23) together to generate a single ultrawide image such as image 2304 shown in FIG. 23. Method 2400 may include one or more feedback loops. For example, method 2400 may include a loop 2412 related to checking 2408, wherein if it is determined that all positions have not been captured (e.g., “NO” at checking 2408), the process flow reverts to a prior point such as setting 2404 the position of the fixation target(s)/graphic(s). If “YES” at checking 2408, the process flow simply proceeds to stitching 2410.

The aspects shown and described in connection with FIGS. 14-24 are not limited to eye imaging, and may be applied to imaging of other objects such as other body parts. For example, the adjusting of imaging components associated with the dual anterior techniques shown and described in FIGS. 14-19 may be applied to achieve imaging of different portions of ears, etc. Similarly, the stitching processes described in connection with FIG. 23 may be utilized to stitch images obtained of other objects.

FIG. 25 is a block diagram schematically illustrating a system (e.g., a computing system) in accordance with one aspect of the disclosure. FIG. 25 illustrates a simplified block diagram of a computing system 2500 for implementing the methods described herein. As illustrated in FIG. 25, the computing system 2500 may be configured to implement at least a portion of the tasks associated with the disclosed methods herein, such as controlling an imaging system such as an OCT system as described herein. For example, system 2500 may be one configuration of the overall system shown in FIG. 1. Computer system 2500 may include a computing device 2502 that may be the same as or similar to external system 112 shown in FIG. 1, unit 504 shown in FIG. 5, and/or computer 614 shown in FIG. 6. Computer system 2500 may include computing components such as any processors, memories, and/or other electronic components that may be integrated within the imaging devices described herein as part of the imaging devices being a self-contained unit as described herein. In one aspect, computing device 2502 is part of or in operable communication with a server system 2504, which also includes a database server 2506. Computing device 2502 may be in communication with a database 2508 through database server 2506. Database 2508 may be configured to store information such as control programs, data, test results, and the like as described herein. Computing device 2502 may be in operative communication with or part of (i) system 2510 (e.g., an imaging system such as an OCT system), where system 2510 may be the same as or similar to the various imaging systems described and shown herein and (ii) a user computing device 2512 of a user 2514 through a network 2516. User computing device 2512 may be utilized by user 2514 to view/analyze results of the various tests and/or experiments described herein. Network 2516 may be any network that allows local area and/or wide area communication between the devices. For example, network 2516 may allow communicative coupling to the Internet through at least one of many interfaces including, but not limited to, at least one of a network, such as the Internet, a local area network (LAN), a wide area network (WAN), an integrated services digital network (ISDN), a dial-up-connection, a digital subscriber line (DSL), a cellular phone connection, and a cable modem (e.g., see wireless transmission 518 in FIG. 5). User computing device 2512 may be any device capable of accessing the Internet including, but not limited to, a desktop computer, a laptop computer, a personal digital assistant (PDA), a cellular phone, a smartphone, a tablet, a phablet, wearable electronics, smart watch, or other web-based connectable equipment or mobile devices. In other aspects, computing device 2502 is configured to perform a plurality of tasks associated with the operation of a system and/or device incorporating the imaging components and/or other corresponding components described herein including, but not limited to the imaging systems such as an OCT system and/or the various other (e.g., non-OCT) applications/imaging systems as described herein.

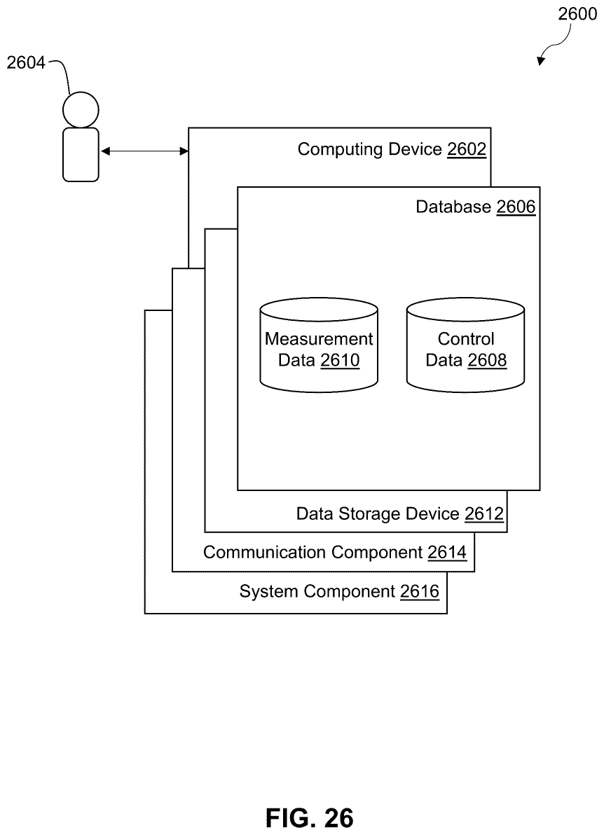

FIG. 26 depicts a component configuration 2600 of computing device 2602 associated with a user 2604. In some aspects, computing device 2602 may be the same as or similar to computing device 2502 shown in FIG. 25 and user 2604 may be the same as or similar to user 2514 shown in FIG. 25. User 2604 may access components of computing device 2602. For example, user 2604 may be a technician/medical professional running an imaging session using an imaging device (e.g., a wearable device) on a subject (e.g., patient) as described and shown herein, such as the various setups shown in FIGS. 10A-10C. Computing device 2602 may also include database 2606 along with other related computing components. In some aspects, database 2606 may be the same as or similar to database 2508 shown in FIG. 25.

In one aspect, database 2606 includes control data 2608 and measurement data 2610. Non-limiting examples of control data 2608 may include control parameters for the various components included in the various imaging devices described and shown herein, such as controls to control laser power, etc. Additional non-limiting examples of suitable control data 2608 include any algorithms and any values of parameters defining the algorithms associated with the disclosed methods as described herein. Measurement data 2610 may include data for aspects such as tracks 142 shown in FIG. 1, and/or measurement data corresponding to the locations of the various lenses and/or other imaging components in the various embodiments described herein. Measurement data 2610 may further include data such as the raw data obtained from the imaging components and that is used to generate images such as shown in FIGS. 3, 18, and 23, for example. This may include other measurement results from the imaging devices described and shown herein, such as other image data, composite image data, output images, and the like. For example, measurement data 2610 may be used as training data to train a model configured to perform image stitching as described herein. Measurement data 2610 may also include any algorithms described herein, and likewise an instructions to execute the various software loops described herein.

Computing device 2602 also includes a number of components that perform specific tasks. In the example aspect, computing device 2602 includes data storage device 2612, communication component 2614, and system component 2616. Data storage device 2612 is configured to store data received or generated by computing device 2602, such as any of the data stored in database 2606 or any outputs of processes implemented by any component of computing device 2602. Communication component 2614 is configured to enable communications between computing device 2602 and other devices (e.g., user computing device 2512 and system 2510, shown in FIG. 25) over a network, such as network 2516 (shown in FIG. 25), or a plurality of network connections using predefined network protocols such as TCP/IP (Transmission Control Protocol/Internet Protocol). System component 2616 is configured to control aspects relating to the imaging devices and/or their imaging systems (e.g., OCT systems) described and shown herein, including but not limited to design, testing, modeling, and/or fabrication parameters. Components 2612-2616 may be a combination of software modules and/or corresponding hardware components, which in some aspects may be dedicated hardware components such as dedicated processors and the like.

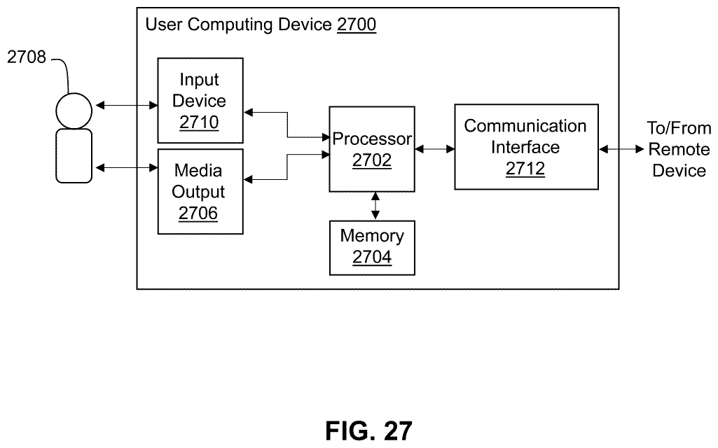

FIG. 27 depicts a configuration of a remote or user computing device 2700, such as, but not limited to, user computing device 2512 (shown in FIG. 25). Computing device 2700 may include a processor 2702 for executing computer-readable/-executable instructions. In some aspects, executable instructions may be stored in a memory area of memory 2704. Processor 2702 may include one or more processing units (e.g., in a multi-core and/or parallel configuration). Memory 2704 may be any device allowing information such as executable instructions and/or other data to be stored and retrieved. Memory 2704 may include one or more computer-readable media (e.g., hard drive, RAM, ROM, and the like).

Computing device 2700 may also include at least one media output component 2706 for presenting information to a user 2708. Media output component 2706 may be any component capable of conveying information to a user 2708. In some aspects, media output component 2706 may include an output adapter, such as a video adapter and/or an audio adapter. An output adapter may be operatively coupled to processor 2702 and operatively coupled to an output device such as a display device (e.g., a liquid crystal display (LCD), organic light emitting diode (OLED) display, cathode ray tube (CRT), or “electronic ink” display) or an audio output device (e.g., a speaker or headphones). In some aspects, media output component 2706 may be configured to present an interactive user interface (e.g., a web browser or client application) to user 2708.

In some aspects, computing device 2700 may include an input device 2710 for receiving input from user 2708. Input device 2710 may include, for example, a keyboard, a pointing device, a mouse, a stylus, a touch sensitive panel (e.g., a touch pad or a touch screen), a camera, a gyroscope, an accelerometer, a position detector, and/or an audio input device. A single component such as a touch screen may function as both an output device of media output component 2706 and input device 2710.

Computing device 2700 may also include a communication interface 2712, which may be communicatively coupled to a remote device. Communication interface 2712 may include, for example, a wired or wireless network adapter or a wireless data transceiver for use with a mobile phone network (e.g., Global System for Mobile communications (GSM), 3G, 4G or Bluetooth) or other mobile data network (e.g., Worldwide Interoperability for Microwave Access (WIMAX)).

Communication interface 2712 may be configured for providing a user interface to user 2708 via media output component 2706 and, optionally, receiving and processing input from input device 2710. A user interface may include, among other possibilities, a web browser and client application. Web browsers enable users 2708 to display and interact with media and other information typically embedded on a web page or a website from a web server. A client application allows users 2708 to interact with a server application associated with, for example, a vendor or business.

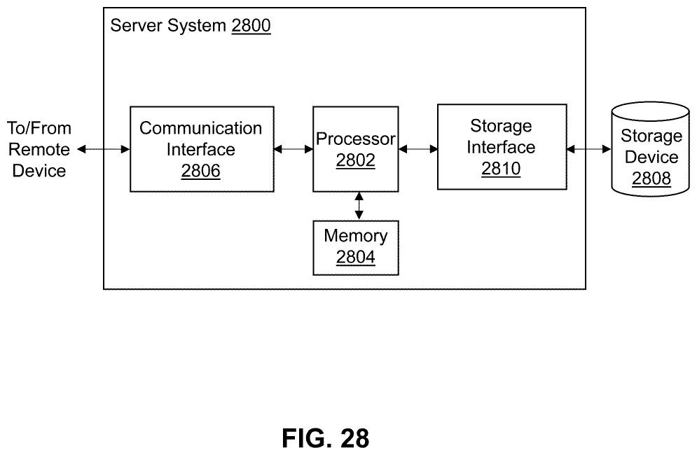

FIG. 28 illustrates an example configuration of a server system 2800. Server system 2800 may include, but is not limited to, database server 2506 and computing device 2502 (both shown in FIG. 25), computing device 2602 shown in FIG. 26, and/or other computer devices as described herein. In some aspects, server system 2800 is the same as or similar to server system 2504 (shown in FIG. 25). Server system 2800 may include a processor 2802 for executing instructions. Instructions may be stored in a memory area of memory 2804, for example. Processor 2802 may include one or more processing units (e.g., in a multi-core or parallel configuration).

Processor 2802 may be operatively coupled to a communication interface 2806 such that server system 2800 may be capable of communicating with a remote device such as user computing device 2512 (shown in FIG. 25) or one or more other server systems 2800. For example, communication interface 2806 may receive requests from user computing device 2512 via a network 2516 (shown in FIG. 25).

Processor 2802 may also be operatively coupled to a storage device 2808. Storage device 2808 may be any computer-operated hardware suitable for storing and/or retrieving data. In some aspects, storage device 2808 may be integrated in server system 2800. For example, server system 2800 may include one or more hard disk drives as storage device 2808. In other aspects, storage device 2808 may be external to server system 2800 and may be accessed by a plurality of server systems 2800. For example, storage device 2808 may include multiple storage units such as hard disks or solid state disks in a redundant array of inexpensive disks (RAID) configuration. Storage device 2808 may include a storage area network (SAN) and/or a network attached storage (NAS) system.

In some aspects, processor 2802 may be operatively coupled to storage device 2808 via a storage interface 2810. Storage interface 2810 may be any component capable of providing processor 2802 with access to storage device 2808. Storage interface 2810 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 2802 with access to storage device 2808.

Memories 2704 (shown in FIGS. 27) and 2804 may include, but are not limited to, non-transitory random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are example only, and are thus not limiting as to the types of memory usable for storage of a computer program.

### Wearable Eyepiece

Additional aspects of the wearable eyepieces as described and shown in the figures are detailed below. For example, with reference to FIG. 1, FIG. 1 depicts an OCT system with the sample arm connected to a wearable eyepiece that implements, among other functions, a mechanism to direct the imaging beam towards both eyes of the wearer to perform OCT imaging. In this embodiment, the light is split into two paths by way of a 50/50 optical coupler (e.g., 132 shown in FIG. 1), after which a specified amount of optical delay (e.g., 134 shown in FIG. 1) (e.g., 3 mm) is introduced to make one path longer than the other. The split light then passes through two free space optical systems that may include a collimation lens (e.g., 136 shown in FIG. 1), a scanning mirror (e.g., 138 shown in FIG. 1), and two relay lenses (e.g., 140 shown in FIG. 1). This optical system focuses light onto either the retina or anterior segment of each eye as described in accordance with certain embodiments herein, allowing for simultaneous high-resolution imaging of both eyes. The collimation lens (e.g., 136 shown in FIG. 1) may also be implemented as a liquid lens with an electronically variable focal length to allow for the accommodation of subjects with different diopters.

In all configurations, the light originating from the sample arm of the OCT system may be split into sub-arms (e.g., two sub-arms), with both paths having a relative length difference to introduce optical delay. Since the images of both eyes will be captured by a single OCT system, the optical delay prevents the two back reflected signals from overlapping when recombined in the optical coupler. Due to the nature of OCT implemented as an interferometer as described herein, signals that travel different distances relative to the length of the reference arm will appear at different axial depths in the final image. For example, FIG. 3 depicts this effect with the cross-sectional images of two retinas layered at different axial depths due to the artificial addition of optical delay. The single image may be split into its two constituent images via a post processing algorithm. In one embodiment, the delay may be implemented as optical fibers with different lengths, where the length difference may be 3 mm. The amount of optical delay introduced in one arm may be chosen to provide sufficient separation distance in the final image such that no overlapping of the two images occur. Additionally, the amount of optical delay should be large enough to prevent overlapping, but not so large as to exceed the axial imaging range of the OCT system. By utilizing optical delay, the device avoids duplication of any part of the core OCT system and allows the images of both eyes to be captured simultaneously in a single image.

After being split into two paths with different delays, the light in each path is focused into or onto the eye by a set of lenses. In some embodiments, the light may be coupled into free space from an optical fiber, in which case it will be divergent, requiring a collimation lens to produce a collimated beam. Taking retinal imaging as an example, the light may be scanned across the retina by a pivotable mirror which, in some embodiments, may appear directly after the collimating lens. In order for light to enter the aperture of the eye at all scanning angles, the pivot position of the mirror has a conjugate position at the pupil of the eye. This can be achieved by including two stages of relay lenses with specified focal lengths and at appropriate distances from each other to allow for the beam to be focused onto the retina while simultaneously allowing for the scanning beam to pivot at the pupil.

In all embodiments, the light directed to each eye may be scanned across an area of the sample by way of a mechanically actuated mirror or beam steering device, as depicted in FIGS. 1, 4, 5, 6, 7, 8, and/or 9. At each point on the sample, the OCT system will retrieve a depth profile of that location, and by scanning the beam across a two-dimensional area, a three-dimensional dataset may be acquired. The scanning mechanisms described herein may be implemented as one or more mechanically pivotable mirrors that can redirect the light along some direction that is not parallel to the central axis of the optical system. For example, a single bonded MEMS mirror from Mirrorcle, Inc. may be used, which can be electrostatically actuated in two dimensions with a maximum angle in each dimension of 5 degrees. It is understood that in all embodiments, two such scanning mirror devices may be implemented, one for each eye.

Referring back to FIG. 1, in FIG. 1, it is depicted that the dual lens systems including the scanning mirror may be movable along tracks (e.g., 142 shown in FIG. 1) or some other motorized mechanism in one or two dimensions to laterally displace the axial paths to each eye. This mechanism may account for anatomical differences between patients such as head size and distance between eyes. It may also be incorporated with other functions such as eye tracking to automatically realign the imaging system in real time to compensate for any coarse movement of the patient's eyes.

FIG. 1 depicts one embodiment in which the majority of the OCT system excluding the sample arm optics is located outside of the eyepiece device. In general, any part of this external system may be incorporated into or mounted onto the eyepiece, providing a more portable setup. Referring back to FIG. 5, FIG. 5 depicts one embodiment in which the laser (e.g., 506 in FIG. 5), DAQ system (e.g., 508 in FIG. 5), and FPGA (e.g., 510 in FIG. 5) for data processing are mounted onto the rear of the eyepiece such that the entire imaging system is self-contained. While unit 504 is shown separate from eyepiece 514 in FIG. 5, all of part of unit 504 may be integrated into eyepiece 514 to further enhance portability and applications for positions such as shown in FIGS. 10A-10C that conventional systems are unable to accommodate. That is, in any of the embodiments, design optimizations may be made such that the wearable device(s) are self-contained portable units. Power supplies (e.g., batteries, etc., not shown) may be incorporated therein as needed. The processed data may be wirelessly transmitted to an external computer or visualization tool by way of Bluetooth or Wi-Fi or other wireless transmission protocol as described herein. It is understood that any or all of the OCT subsystems such as the optical splitters, circulators, reference arm, MZI, or photodetectors as described herein may be implemented in such a way that they can be compactly incorporated into the eyepiece. For example, these subsystems may be implemented on a PIC chip as described herein to drastically reduce the physical size and/or weight of the overall device.

As depicted in FIG. 9, the various eyepieces described herein may include a screen or projected surface capable of displaying video media, text, images, or visual cues and instructions for imaging, where applicable. In some embodiments, this display may be implemented as one or two liquid crystal displays (LCD), LED screens and/or as a semi-reflective surface onto which images are projected by one or more small projectors. In any case, this display surface may show two images, one for each eye, positioned in such a way that the media appears to be a single image to the viewer/patient. If dynamic media such as a video or movie is displayed, the patient may physically move their eye(s) to view different locations of the screen. This will have the effect of distributing the OCT imaging beam across different regions of the retina or anterior segment, in turn allowing the final image to have a large field of view without requiring the scanning mechanism to project the imaging beam at extreme angles or positions.

The eyepieces described herein may include one or more cameras (e.g., a set of cameras) included for the purpose of tracking the patient's eye position or for estimating their point of visual focus, such as cameras 144 shown in FIG. 1, and/or in other figures herein. Additionally, a set of LEDs or other illumination devices may be included to allow for the eye tracking cameras to accurately capture images of the eye. Alternatively, a media display screen may also provide sufficient illumination for the eye tracking cameras to function properly. In any case, the cameras may be connected to a feedback loop associated with a motorized translation mechanism to align the imaging axis with the patient's eye, which may vary based on coarse eye movement or anatomical differences between patients.

In some embodiments, a fundus camera may be included which is capable obtaining a 2D photographic image of the retina, which can be used for alignment instead of, or in conjunction with eye tracking cameras which identify the pupil position. Fundus cameras may need an additional light source to provide flash illumination when obtaining a photograph of the retina.

The eyepiece may be constructed such that it securely fits on the subject's (e.g., patient's) head and is impervious to movement. This may include a light and compact construction of the system components, along with attachment mechanisms such as straps that wrap around the wearer's head such as strap(s) 110 shown in FIG. 1.

### Software and Eye-Tracking

Additional aspects of the software and eye-tracking aspects described herein are outlined below. As described above, FIG. 11, for example, depicts a flowchart outlining a process flow (e.g., a software control loop) for an imaging session, including the processing of eye position information from the eye tracking system for aligning the imaging beam, and controlling hardware in the imaging (e.g., OCT) system to optimize image quality according to the various applicable embodiments herein. FIGS. 12A and 12B are diagrams illustrating how an eye tracker ascertains pupil position and helps guide an incident beam through the pupil after the users move their eye, according to applicable embodiments described herein.

In some embodiments, the wearable eyepiece may include a screen to display media to the patient as described herein. In this scenario, the software will also be responsible for loading media to the screen. The screen may display media which is designed to promote a certain type of gaze and/or attract the gaze of the patient to specific positions. In this case, the software may be able to synchronize the positions of objects of interest within this media with the eye tracking system to provide additional information and control over the distribution of images acquired across the retina.

Further regarding the display of media content and stitching, in one scenario media content images may follow a grid pattern to enable streamlined stitching. For example, media content images may be arranged on the display in a similar arrangement to that shown for images 2300 shown in FIG. 23, to provide for straightforward stitching downstream. In another scenario, the media content images may follow a “random” (e.g., non-rectilinear or non-grid like) pattern during a piece of media content (e.g., entertainment) such as a cartoon or game. In this scenario, eye tracking (e.g., pupil) cameras determine the gaze location of the subject and in conjunction with analysis of images already acquired within a widefield acquisition, including updating the point of fixation within the media content until full coverage of the widefield image is obtained. Additionally, the gaze information obtained from the eye tracking (e.g., pupil tracking) cameras may be used to inform an image stitching algorithm by providing relative locations of each constituent sub-image, loosening the requirement for overlap between images which is normally required for correlation-based stitching.

In some embodiments, the functions of the eye tracking cameras may be replaced by or work together with fundus cameras to perform automatic alignment of the imaging beams.

The software may also implement a secure database system to keep track of patient records and associated images such as shown in and described in connection with FIGS. 25-28. Such a database may also be capable of saving specific parameters related to each subject (e.g., each patient), component parameters such as diopter parameters for liquid lens calibration, blinking frequency, relative distribution of eye movement, etc. The specific setting related to each patient may be loaded from the database for repeated imaging sessions. This database may be part of or otherwise in communication with system 2508 (shown in FIG. 25, described in more detail herein) and/or database 2606 and/or linked in connection with control data 2608 (both shown in FIG. 26, described in more detail herein). For example, linking the secure database system serves to connect the saving and reuse of metadata for patients to control the OCT machine, for embodiments described herein where patient control is enabled.

In some embodiments, to enable the functional OCT image acquisition, the OCT images will be processed with a split-spectrum amplitude decorrelation algorithm and discrete Fourier transform registration to create angiography images. Standard deviations, cross-correlation, and power spectrum analysis over the signal fluctuations can be used to create dynamic contrast images.

### Additional Aspects and Applications

In some embodiments, multiple sample arms may be implemented. For example, an imaging device as described herein may be outfit with two or more sample arms.

In some embodiments, an eyepiece such as that depicted in FIGS. 1, 4, 5, 7, and/or 9 may implement an (e.g., ophthalmic) imaging method other than OCT. For example, scanning laser ophthalmoscopy (SLO) operates in a similar fashion to OCT by scanning a laser beam across the retina. In this case, much of the same materials and methods for scanning, focusing, eye tracking, media display, and/or mechanical design outlined herein can be used to implement a wearable SLO device. Additionally, a wearable imaging device as described herein may implement, in place of or in addition to OCT/SLO, a confocal microscope system, such as for realizing imaging beyond eye imaging.

Additional adaptations to the imaging device described herein may be made to facilitate imaging of other portions of the body, such as dental imaging, where an imaging system may include an imaging device configured to facilitate imaging of the top and bottom rows of teeth simultaneously using the simultaneous capture techniques described herein. For example, the methods described herein for simultaneously acquiring images of both eyes, ears, rows of teeth, etc. may be applied to scenarios outside of ophthalmic, ear, and/or dental imaging, and more generally for imaging of more than two objects at one time, as described in the additional embodiments and/or applications below. For example, the simultaneous imaging techniques described herein may be utilized, configured, and implemented for applications such as imaging of a plurality of samples/objects, such as in connection with samples of 96-well plates (e.g., imaging of multiple samples simultaneously in a 96-well plate).

Additionally, certain aspects of the wearable devices described herein may be configured to be controllable by the subject. For example, based on an input from the subject (e.g., from voice input, gaze tracking, blinking, and/or other input devices such as a handheld remote control), the subject can control the device by on their own. This may enable self-guided testing/imaging. For example, in connection with evaluating 1106 shown in FIG. 5, blinking can be encoded as a control mechanism, and processed separate from non-coded blinking. That is, the system may be configured (e.g., via one or more cameras such as cameras 1200/1210 shown in FIG. 12) to recognize a certain pattern of blinks as a control command. For example, three consecutive blinks may be recognized as a control command, whereas a normal single blink would be disregarded (e.g., not construed as a control command).

This written description uses examples to disclose the invention, including the best mode, and also to enable any person skilled in the art to practice the invention, including making and using any devices or systems and performing any incorporated methods. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

As used herein, the terms “about,” “substantially,” “essentially” and “approximately” when used in conjunction with ranges of dimensions, concentrations, temperatures or other physical or chemical properties or characteristics is meant to cover variations that may exist in the upper and/or lower limits of the ranges of the properties or characteristics, including, for example, variations resulting from rounding, measurement methodology or other statistical variation.

When introducing elements of the present disclosure or the embodiment(s) thereof, the articles “a”, “an”, “the” and “said” are intended to mean that there are one or more of the elements. The terms “comprising,” “including,” “containing” and “having” are intended to be inclusive and mean that there may be additional elements other than the listed elements. The use of terms indicating a particular orientation (e.g., “top”, “bottom”, “side”, etc.) is for convenience of description and does not require any particular orientation of the item described.

As various changes could be made in the above constructions and methods without departing from the scope of the disclosure, it is intended that all matter contained in the above description and shown in the accompanying drawing(s) shall be interpreted as illustrative and not in a limiting sense. Aspects of the various embodiments may be combined.

What is claimed is:

1. An imaging system for dual site imaging comprising:
a wearable device configured to be worn by a subject, the wearable device comprising a set of imaging components including:
a light source; and
a sample arm including one or more lenses and one or more scanning mirrors; and
a computing device communicatively coupled to the wearable device, the computing device comprising at least one processor and at least one memory in communication with the at least one processor, the at least one processor programmed to:
execute, while the subject is wearing the wearable device, a dual site image data acquisition operation configured to control the set of imaging components to simultaneously obtain (i) first image data of a first body portion of a body of the subject and (ii) second image data of a second body portion of the subject's body;
execute a dual site image data processing operation on the first image data and the second image data; and
generate, based on an output from the dual site image data processing operation, one or more images depicting the first body portion and the second body portion.

2. The imaging system of claim 1, wherein the set of imaging components comprises one of an optical coherence tomography (OCT) system, a scanning laser ophthalmoscopy (SLO) system, or a confocal microscope system.

3. The imaging system of claim 2, wherein the set of imaging components comprises the OCT system, and wherein the OCT system comprises a single OCT system.

4. The imaging system of claim 3, wherein the set of imaging components is configured to parallelize an imaging capability of the single OCT system to image both the first body portion and the second body portion of the subject using the single OCT system.

5. The imaging system of claim 1, wherein the sample arm includes two sub-arms, and the set of imaging components is configured to cause light originating from the sample arm to be split into the two sub-arms, each sub-arm including a respective path, the paths having a relative length difference to introduce optical delay.

6. The imaging system of claim 5, wherein the set of imaging components further includes an optical coupler, and the optical delay is configured to prevent back reflected light from overlapping when recombined in the optical coupler.

7. The imaging system of claim 6, wherein the one or more images includes a final image including a first image based on the first image data and a second image based on the second image data, and an amount of the optical delay is selected and introduced in one of the sub-arms to provide separation distance in the final image such that no overlapping of the first image and the second image is present in the final image.

8. The imaging system of claim 1, wherein the one or more images includes a plurality of sub-images and the at least one processor is further programmed to:
stitch the sub-images into a single ultrawide image.

9. The imaging system of claim 1, wherein the wearable device is configured as a self-contained unit to accommodate use of the wearable device: (i) on the subject positioned in a Fowler's position; (ii) on the subject positioned in a supine position; and (iii) on the subject positioned in a prone position.

10. The imaging system of claim 1, wherein the wearable device is configured with one or more display screens.

11. The imaging system of claim 10, wherein the wearable device is a head-mountable device configured for wearing on a head of the subject and the one or more display screens are configured to display visual content for viewing by the subject.

12. The imaging system of claim 11, wherein the visual content is displayed within the head-mountable device on the one or more display screens, and the visual content is displayed for viewing by the subject while the subject is awake and in a state of active engagement with the visual content.

13. The imaging system of claim 1, wherein the wearable device is a head-mountable device configured for wearing on a head of the subject and configured with one or more audio elements.

14. The imaging system of claim 13, wherein the one or more audio elements are configured to output audible content for listening by the subject during an imaging session.

15. The imaging system of claim 1, wherein the wearable device is a head-mountable device configured for wearing on a head of the subject and the at least one processor is further programmed to:
receive a control input from the subject for control of one or more operations of the wearable device during an imaging session.

16. The imaging system of claim 1, wherein the first body portion of the subject's body is a first eye of the subject and the second body portion of the subject's body is a second eye of the subject.

17. The imaging system of claim 16, wherein the wearable device is configured to track and register positions of the first eye and the second eye relative to one or more defined imaging positions.

18. The imaging system of claim 17, wherein the wearable device includes one or more display screens and one or more cameras, and wherein the wearable device is configured to, via at least the one or more cameras, track and register a gaze of one eye, from amongst the first eye and the second eye, being guided to one or more different locations associated with one or more corresponding fixation targets displayed on the one or more display screens during imaging of the one eye.

19. The imaging system of claim 1, wherein the first body portion of the subject's body is a first ear of the subject and the second body portion of the subject's body is a second ear of the subject.

20. A dual site imaging method implemented using (i) a wearable device configured to be worn by a subject, the wearable device comprising a set of imaging components including a light source and a sample arm including one or more lenses and one or more scanning mirrors, and (ii) a computing device communicatively coupled to the wearable device, the computing device comprising at least one processor and at least one memory in communication with the at least one processor, the dual site imaging method comprising:
providing the wearable device for wearing by the subject;
executing, while the subject is wearing the wearable device, and by the computing device, a dual site image data acquisition operation configured to control the set of imaging components to simultaneously obtain (i) first image data of a first body portion of a body of the subject and (ii) second image data of a second body portion of the subject's body;
executing, by the computing device, a dual site image data processing operation on the first image data and the second image data; and
generating, based on an output from the dual site image data processing operation, and by the computing device, one or more images depicting the first body portion and the second body portion.
